(19) United States
(12) Patent Application Publication
(10) Pub. No.: US 2013/0072430 A1
(43) Pub. Date: Mar. 21, 2013

(54) METHODS FOR TREATMENT OR PROPHYLAXIS OF KIDNEY OR LIVER DYSFUNCTION

(71) Applicant: NPS Pharmaceuticals, Inc., Bedminster, NJ (US)

(72) Inventors: Bo Joelsson, Mountain Lakes, NJ (US); Henry H. Chu, Bedminster, NJ (US)

(73) Assignee: NPS PHARMACEUTICALS, INC., Bedminster, NJ (US)

(21) Appl. No.: 13/673,386

(22) Filed: Nov. 9, 2012

Related U.S. Application Data
(63) Continuation-in-part of application No. PCT/US11/36106, filed on May 11, 2011.
(60) Provisional application No. 61/333,678, filed on May 11, 2010.

(51) Int. Cl. A61K 38/26

(52) U.S. Cl. CPC A61K 38/26
USPC 514/11.7

(57) ABSTRACT

Methods are provided for treatment or prophylaxis of liver or kidney dysfunction in individuals experiencing one or more of intestinal failure, short bowel syndrome or parenteral nutrition by the administration of GLP-2 or GLP-2 analogs.

### CROSS-REFERENCE TO RELATED APPLICATIONS

This application is a continuation-in-part of copending International Application No. PCT/US2011/036106, titled METHODS FOR TREATMENT OR PROPHYLAXIS OF KIDNEY OR LIVER DYSFUNCTION, with an international filing date of May 11, 2011, which claims the benefit of U.S. Provisional Application No. 61/333,678, filed May 11, 2010, both of which are hereby incorporated by reference in their entireties.

### TECHNICAL FIELD

The invention relates to methods useful for treatment or prophylaxis of liver or kidney dysfunction commonly associated with parenteral nutrition, short bowel syndrome and intestinal failure. More particularly, the invention relates to methods of using of a GLP-2 peptide, or analogs thereof, for the treatment or prophylaxis of liver or kidney dysfunction commonly associated with parenteral nutrition, short bowel syndrome and intestinal failure.

### BACKGROUND

Intestinal failure is a condition caused “by the critical reduction of functional gut mass below the minimal amount necessary for adequate digestion and absorption to satisfy body nutrient and fluid requirements” (Goulet et al., Curr. Op. Org. Trans., 14:256, 2009). This condition is often caused by short bowel syndrome (SBS), although it can also result from other insults on the gut (Thompson et al., J. Am. Coll. Surg. 201:85, 2005). Intestinal failure can be managed to some extent through parenteral nutrition (PN): the provision of nutrition intravenously as opposed to through the gastrointestinal tract (Klein, Gastroenterology 121:970, 2002).

In patients with SBS, a process called intestinal adaptation, in which the short bowel increases its absorption to compensate for the lost tissue, sometimes restores enough gut function that the patient can cease PN after limited treatment (Welters et al., ANZ J. Surg. 72:229, 2002). However, other individuals depend on long term or chronic PN as a source of nutrition. The availability of PN to patients with intestinal failure is limited, however, by PN-associated liver and kidney dysfunction.

PN-associated liver disease (PNALD) occurs to some degree in most patients receiving long-term PN (Cavicchi et al., Ann. Int. Med. 132:525, 2000) (Salvino R, J. Parenter Enteral Nutr. 30:202, 2006). In some cases it can be progressive resulting in hepatic failure necessitating liver transplantation (Buchman et al., Hepatology 43:9, 2006) (Chan et al., Surgery 126:28, 1999). Although the frequency of severe, irreversible liver injury from PNALD varies depending on circumstances, some reports suggest that it will ultimately occur in more than 50% of long-term PN recipients. Furthermore, PN-associated liver dysfunction impairs the process of intestinal adaptation in short bowel patients, which may necessitate more PN (Goulet et al., Curr. Op. Org. Trans., 14:256, 2009).

PNALD pathogenesis and progression factors are incompletely understood, and reliable parameters to identify patients at high risk for progression are lacking (Fulford et al., Nutr. Clin. Pract. 19:274, 2004) (Buchman, Gastroenter. 130:S5, 2006). However, SBS due to massive intestinal resection has been identified as one of the risk factors of PNALD, as interruption in enterohepatic circulation causes alterations in bile acid metabolism and excretion.

Another important and serious complication in long-term PN patients is renal dysfunction, manifested by a progressive decrease in creatinine clearance. In a series of long term PN patients, creatinine clearance was found to decrease on average 3.5% per year with a follow-up of 10 years or greater (Buchman et al., J. Parenter Enteral Nutr. 17:438, 1993). The decrease could not be ascribed to advancing age, nephrotoxic drug use, nutritional status, amino acid content of PN or septicemia episodes. Similarly creatinine clearance was found to be reduced in children receiving long term PN, with the degree of impairment directly proportional to the duration of PN (Moukarzel et al., J. Pediatr. 119:864, 1991). No evidence for tubular dysfunction, nephrocalcinosis or obstructive uropathy was found in the children studied.

Unfortunately, patients with SBS may be dependent on long-term PN and therefore have limited options if liver or renal dysfunction or failure develops. Patients who develop irreversible and severe reductions in renal function may progress to renal failure, necessitating chronic dialysis or renal transplantation. Those patients with SBS who develop irreversible liver injury and failure, may be referred for intestine or combined intestine-liver transplantation (Buchman et al., Gastroenterololgy 124:1111, 2003) (Keller et al., Best Prac. Res. Clin Gastroenterol. 18: 977, 2004) (Chungfat et al., J. Amer. Coll. Surg. 205:755, 2007).

Recent developments in PN formulations may provide some relief from PNALD. In several small studies, substitution of all or some of the plant based lipids for fish based lipids in PN formulations resulted in an increase in liver function in individuals receiving PN (Antébi et al., 2004, Mertes et al., Ann. Nutr. Metab. 50:253, 2006). However, the long term safety and efficacy of these formulations have not been established (Wiles and Woodward, Curr. Op. Clin. Nutr. Metab. Care 21:265, 2009), further underscoring the need for additional treatments for PNALD.

### BRIEF DESCRIPTION

The present invention provides methods for treating kidney or liver dysfunction in individuals. In some instances, the methods are used with individuals receiving parenteral nutrition, experiencing short bowel syndrome, or suffering from intestinal failure. The methods disclosed comprise the step of administering to an individual a GLP-2 peptide or a GLP-2 peptide analog in an amount effective to treat liver or kidney disease.

This invention also provides methods for prophylaxis against kidney or liver dysfunction in individuals. In some instances, the methods are used with individuals receiving parenteral nutrition, experiencing short bowel syndrome, or suffering from intestinal failure. The methods disclosed comprise administering to an individual a GLP-2 peptide, or a GLP-2 peptide analog in an amount effective for prophylaxis of liver or kidney disease.

In one embodiment, the invention is directed to a method of treating impaired liver function in an individual experiencing intestinal failure, short bowel syndrome, or parenteral nutrition, comprising the step of administering to an individual having impaired liver function one or more of a GLP-2 peptide or a GLP-2 peptide analog in an amount effective to cause improvement in liver function.

In one such embodiment, the GLP-2 peptide or the GLP-2 peptide analog is administered at a dose of between about 0.001 mg/kg/day and about 10 mg/kg/day. In another such embodiment, the GLP-2 peptide analog is teduglutide (SEQ ID NO.:4). In another such embodiment, teduglutide is administered at a dose of between about 0.05 mg/kg/day and about 0.1 mg/kg/day.

In one such embodiment, the improvement in liver function is observed within about four weeks after the beginning of administering the GLP-2 peptide or GLP-2 peptide analog to the individual.

In one such embodiment, the improvement in liver function is monitored by the use of one or more diagnostic biomarkers. In another such embodiment, the diagnostic biomarkers are selected from the group consisting of: bilirubin, gamma glutamyl transferase, alanine transaminase, aspartate aminotransferase, alkaline phosphatase and albumin.

In another such embodiment, the individual level of bilirubin, gamma glutamyl transferase, alanine transaminase, aspartate aminotransferase or alkaline phosphatase, if selected, decreases at least about 5 percent. In another such embodiment, the level of albumin, if selected, increases at least about 5 percent.

In a separate embodiment, the invention is directed to a method for prophylaxis against impairment of liver function in an individual experiencing intestinal failure, short bowel syndrome, or parenteral nutrition, comprising the step of administering to an individual one or more of a GLP-2 peptide or a GLP-2 peptide analog in an amount effective for prophylaxis against impaired liver function.

In one such embodiment, the GLP-2 peptide or the GLP-2 peptide analog is administered at a dose of between about 0.001 mg/kg/day and about 10 mg/kg/day. In another such embodiment, the GLP-2 peptide analog is teduglutide (SEQ ID NO.:4). In another such embodiment, teduglutide is administered at a dose of between about 0.05 mg/kg/day and about 0.1 mg/kg/day.

In one such embodiment, the prophylaxis against impaired liver function is observed within about four weeks after the beginning of administering the GLP-2 peptide or GLP-2 peptide analog to the individual.

In one such embodiment, the prophylaxis against impaired liver function is monitored by the use of one or more diagnostic biomarkers. In another such embodiment, the diagnostic biomarkers are selected from the group consisting of: bilirubin, gamma glutamyl transferase, alanine transaminase, aspartate aminotransferase, alkaline phosphatase and albumin.

In another such embodiment, the individual level of bilirubin, gamma glutamyl transferase, alanine transaminase, aspartate aminotransferase or alkaline phosphatase, if selected, increases, if at all, less than about 10 percent. In another such embodiment, the level of albumin, if selected, decreases, if at all, less than about 10 percent.

In a separate embodiment, the invention is drawn to a method of treating impaired kidney function in an individual experiencing intestinal failure, short bowel syndrome, or parenteral nutrition, comprising the step of administering to an individual having impaired kidney function one or more of a GLP-2 peptide or a GLP-2 peptide analog in an amount effective to cause improvement in kidney function.

In one such embodiment, the GLP-2 peptide or the GLP-2 peptide analog is administered at a dose of between about 0.001 mg/kg/day and about 10 mg/kg/day. In another such embodiment, the GLP-2 peptide analog is teduglutide (SEQ ID NO.:4). In another such embodiment, teduglutide is administered at a dose of between about 0.05 mg/kg/day and about 0.1 mg/kg/day.

In one such embodiment, the improvement in kidney function is observed within about four weeks after the beginning of administering the GLP-2 peptide or GLP-2 peptide analog to the individual.

In one such embodiment, the improvement in kidney function is monitored by the use of one or more diagnostic biomarkers. In another such embodiment, the diagnostic biomarkers are selected from the group consisting of: urea nitrogen, creatinine and glomerular filtration rate. In another such embodiment, the individual level of urea nitrogen, or creatinine, if selected, decreases at least about 5 percent. In another such embodiment, the level of glomerular filtration rate, if selected, increases at least about 5 percent.

In a separate embodiment, the invention is drawn to a method for prophylaxis against impairment of kidney function in an individual experiencing intestinal failure, short bowel syndrome, or parenteral nutrition, comprising administering to an individual one or more of a GLP-2 peptide or a GLP-2 peptide analog in an amount effective for prophylaxis against impaired kidney function.

In one such embodiment, the GLP-2 peptide or the GLP-2 peptide analog is administered at a dose of between about 0.001 mg/kg/day and about 10 mg/kg/day. In another such embodiment, the GLP-2 peptide analog is teduglutide (SEQ ID NO.:4). In another such embodiment, teduglutide is administered at a dose of between about 0.05 mg/kg/day and about 0.1 mg/kg/day.

In one such embodiment, the prophylaxis against impaired kidney function is observed within about four weeks after the beginning of administering the GLP-2 peptide or GLP-2 peptide analog to the individual.

In one such embodiment, the prophylaxis against impaired kidney function is monitored by the use of one or more diagnostic biomarkers. In another such embodiment, the diagnostic biomarkers are selected from the group consisting of: urea nitrogen, creatinine and glomerular filtration rate. In another such embodiment, the individual level of urea nitrogen, or creatinine, if selected, increases, if at all, less than about 5 percent. In another such embodiment, the level of glomerular filtration rate, if selected, decreases, if at all, less than about 5 percent.

### BRIEF DESCRIPTION OF THE DRAWINGS

FIG. 1 is a graph which depicts the average change from baseline over time of alanine transaminase (ALT) in subjects treated with varying doses of teduglutide or placebo.

FIG. 2 is a graph which depicts the average change from baseline over time of aspartate aminotransferase (AST) in subjects treated with varying doses of teduglutide or placebo.

FIG. 3 is a graph which depicts the average change from baseline over time of total bilirubin in subjects treated with varying doses of teduglutide or placebo.

FIG. 4 is a graph which depicts the average change from baseline over time of alkaline phosphatase (ALP) in subjects treated with varying doses of teduglutide or placebo.

FIG. 5 is a graph which depicts the average change from baseline over time of ALT in subjects treated with varying doses of teduglutide or placebo, where the subjects had abnormal baseline values of ALT.

FIG. 6 is a graph which depicts the average change from baseline over time of ALT in subjects treated with varying doses of teduglutide or placebo, where the subjects had normal baseline values of ALT.

FIG. 7 is a graph which depicts the average change from baseline over time of AST in subjects treated with varying doses of teduglutide or placebo, where the subjects had abnormal baseline values of AST.

FIG. 8 is a graph which depicts the average change from baseline over time of AST in subjects treated with varying doses of teduglutide or placebo, where the subjects had normal baseline values of AST.

FIG. 9 is a graph which depicts the average change from baseline over time of creatinine in subjects treated with varying doses of teduglutide or placebo.

FIG. 10 is a graph which depicts the average change from baseline over time of urea nitrogen in subjects treated with varying doses of teduglutide or placebo.

FIG. 11 is a graph which depicts the average change from baseline over time of GFR in subjects treated with varying doses of teduglutide or placebo.

FIG. 12 is a graph which depicts the percent of individuals from groups receiving varying doses of teduglutide or placebo that experience a decrease in ALP greater than 5% over time.

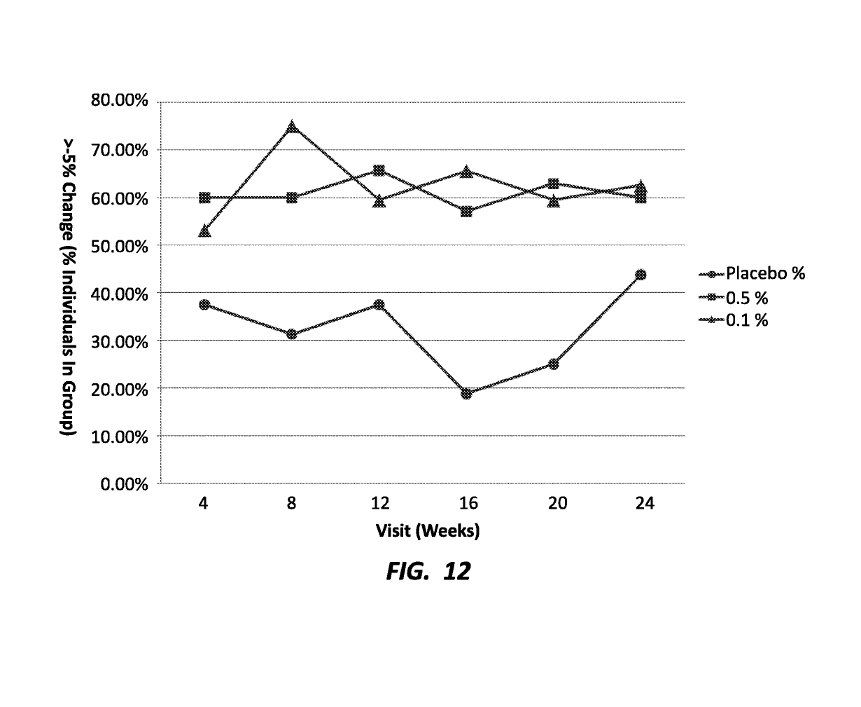

FIG. 13 is a graph which depicts the percent of individuals from groups receiving varying doses of teduglutide or placebo that experience a decrease in ALT greater than 5% over time.

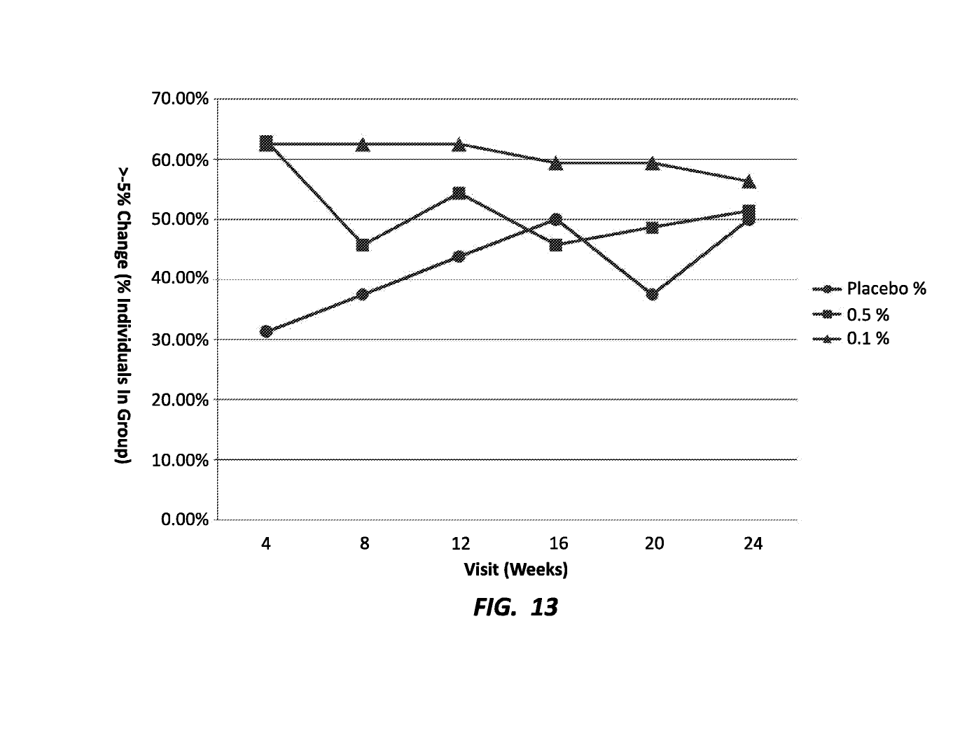

FIG. 14 is a graph which depicts the percent of individuals from groups receiving varying doses of teduglutide or placebo that experience an increase in ALP greater than 5% over time.

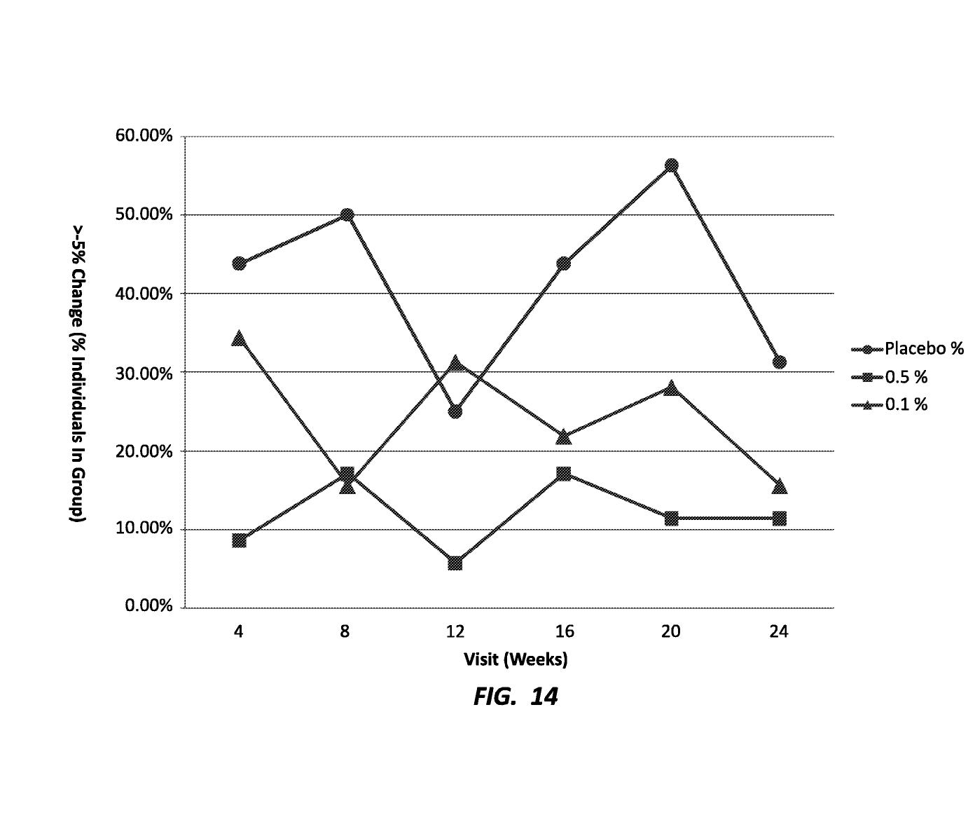

FIG. 15 is a graph which depicts the percent of individuals from groups receiving varying doses of teduglutide or placebo that experience an increase in ALT greater than 5% over time.

FIG. 16 is a graph which depicts the percent of individuals from groups receiving varying doses of teduglutide or placebo that experience a decrease in GFR greater than 5% over time.

FIG. 17 is a graph which depicts the average change from baseline of ALT in individuals treated with placebo or 0.05 mg/kg/day teduglutide.

FIG. 18 is a graph which depicts the average percent change from baseline of ALT in the data depicted in FIG. 17.

FIG. 19 is a graph which depicts the average change from baseline of AST in individuals treated with placebo or 0.05 mg/kg/day teduglutide

FIG. 20 is a graph which depicts the average percent change from baseline of AST in the data depicted in FIG. 19.

FIG. 21 is a graph which depicts the average change from baseline of albumin in individuals treated with placebo or 0.05 mg/kg/day teduglutide

FIG. 22 is a graph which depicts the average percent change from baseline of albumin in the data depicted in FIG. 21.

FIG. 23 is a graph which depicts the average change from baseline of ALP in individuals treated with placebo or 0.05 mg/kg/day teduglutide

FIG. 24 is a graph which depicts the average percent change from baseline of ALP in the data depicted in FIG. 23.

FIG. 25 is a graph which depicts the average change from baseline of bilirubin in individuals treated with placebo or 0.05 mg/kg/day teduglutide

FIG. 26 is a graph which depicts the average percent change from baseline of bilirubin in the data depicted in FIG. 25.

FIG. 27 is a graph which depicts the average change from baseline of gamma glutamyl transferase (GGT) in individuals treated with placebo or 0.05 mg/kg/day teduglutide

FIG. 28 is a graph which depicts the average percent change from baseline of GGT in the data depicted in FIG. 27.

### DETAILED DESCRIPTION

The present invention provides methods for treating kidney or liver dysfunction in individuals. In some instances, the methods are used with individuals receiving parenteral nutrition, experiencing short bowel syndrome, or suffering from intestinal failure. The methods disclosed comprise the step of administering to an individual a GLP-2 peptide or a GLP-2 peptide analog in an amount effective to treat liver or kidney disease.

This invention also provides methods for prophylaxis against kidney or liver dysfunction in individuals. In some instances, the methods are used with individuals receiving parenteral nutrition, experiencing short bowel syndrome, or suffering from intestinal failure. The methods disclosed comprise administering to an individual a GLP-2 peptide, or a GLP-2 peptide analog in an amount effective for prophylaxis of liver or kidney disease.

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

### DEFINITIONS

Unless specifically defined otherwise, the technical terms, as used herein, have their normal meaning as understood in the art. The following terms are specifically defined with examples for the sake of clarity.

The term “GLP-2 peptide” and the term “GLP-2” refer herein to the various naturally produced forms of GLP-2, particularly the mammalian forms, e.g., rat GLP2, ox GLP-2, porcine GLP-2, bovine GLP-2, guinea pig GLP-2, hamster GLP-2 and human GLP-2, the sequences of which have been reported by many authors including Buhl et al in J. Biol. Chem., 263:8621, 1988, which is hereby incorporated by reference in its entirety.

GLP-2 peptides include peptides that conform to the general formula represented below as SEQ ID NO:1:

R1-[Y]m-His-Ala-Asp-Gly-Ser-Phe-Ser-Asp-Glu-Met-
Asn-Thr-aa1-Leu-Ala-aa2-Leu-Ala-aa3-Arg-Asp-Phe-
Ile-Asn-Trp-Leu-aa4-aa5-Thr-Lys-Ile-Thr-Asp-[X]-
n-R2

wherein aa refers to an amino acid residue that is synthetic or genetically encoded, and;

aa1 is a neutral/polar/large/nonaromatic residue such as Ile or Val;

aa2 is a neutral/polar residue such as Asn or Ser;

aa3 is a neutral residue such as Ala or Thr;

aa4 is a neutral/polar/large/nonaromatic residue such as Ile or Leu;

aa5 is a neutral or basic residue such as Gin or His;

X is Arg, Lys, Arg-Lys or Lys-Lys;

Y is Arg or Arg-Arg;

m is 0 or 1;

n is 0 or 1;

R1 is H or an N-terminal blocking group; and

R2 is OH or a C-terminal blocking group SEQ ID NO:1.

The “blocking groups” represented by R1 and R2 are chemical groups that are routinely used to confer biochemical stability and resistance to digestion by exopeptidase. Suitable N-terminal protecting groups include, for example, C₁₋₅ alkanoyl groups such as acetyl. Also suitable as N-terminal protecting groups are amino acid analogs lacking the amino function. Suitable C-terminal protecting groups include groups which form ketones or amides at the carbon atom of the C-terminal carboxyl, or groups which form esters at the oxygen atom of the carboxyl. Ketone and ester-forming groups include alkyl groups, particularly branched or unbranched C₁₋₅ alkyl groups, e.g. methyl, ethyl and propyl groups, while amide-forming groups include amino functions such as primary amine, or alkylamino functions, e.g. mono-C₁₋₅-alkylamino and di-C₁₋₅ alkylamino groups such as methylamino, ethylamino, dimethylamino, diethylamino, methylethylamino and the like. Amino acid analogs are also suitable for protecting the C-terminal end of the present compounds, for example, decarboxylated amino acid analogs such as agmatine.

GLP-2 peptides are known in the art, and are further disclosed in U.S. Pat. No. 5,990,077, which is hereby incorporated by reference in its entirety.

The term “GLP-2 peptide analog” and the term “GLP-2 analog” refer herein to a peptide that incorporates an amino acid substitution at one or more sites within a GLP-2 peptide “background”, which is either a mammalian GLP-2 species per se, or is a variant of a mammalian GLP-2 species in which the C-terminus and/or the N-terminus has been altered by addition of one or two basic residues, or has been modified to incorporate a blocking group of the type used conventionally in the art of peptide chemistry to protect peptide termini from undesired biochemical attack and degradation in vivo. Thus, GLP-2 peptide analogs incorporate an amino acid substitution in the context of any mammalian GLP-2 species, including but not limited to human GLP-2, bovine GLP-2, rat GLP-2, degu GLP-2, ox GLP-2, porcine GLP-2, guinea pig GLP-2 and hamster GLP-2, the sequences of which have been reported by many authors, including Buhl et al, J. Biol. Chem., 1988, 263(18):8621, which is hereby incorporated by reference. The GLP-2 analogs disclosed herein only include peptides that when administered in an effective dose to individuals experiencing one or more of parenteral nutrition, short bowel syndrome or intestinal failure, demonstrate at least one of the following properties: prophylaxis of liver dysfunction, prophylaxis of kidney dysfunction, treatment of liver dysfunction, or treatment of kidney dysfunction. The disclosure provided herein, and the knowledge available in the art would allow a person skilled in the art to determine which of GLP-2 peptides or GLP-2 analogs would retain at least one of the said properties. For example, the disclosure provided herein provides guidance regarding the design of GLP-2 peptides and GLP-2 peptide analogs. Furthermore, the guidance provided herein demonstrate how to determine, using diagnostic biomarkers, whether a GLP-2 peptide or a GLP-2 analog have one of the properties of prophylaxis of liver dysfunction, prophylaxis of kidney dysfunction, treatment of liver dysfunction, or treatment of kidney dysfunction. In this light, the following examples of GLP-2 analogs are provided.

In some instances, GLP-2 peptide analogs according to the present invention include peptides that conform to the sequence of the general formula presented below as SEQ ID NO:2:

R1-(Y1)m-X1-X2-X3-X4-Ser5-Phe6-Ser7-Asp8-(P1)-
Leu14-Asp15-Asn16-Leu17-Ala18-X19-X20-Asp21-
Phe22-(P2)-Trp25-Leu26-Ile27-Gln-28-Thr29-Lys30-
(P3)-(Y2)n-R2,

wherein

X1 is His or Tyr

X2 is Ala or any other amino acid conferring on said analog resistance to dipeptidyl peptidase IV enzyme;

X3 is Asp or Glu;

X4 is Gly or Ala;

P1 is Glu-X10-Asn-Thr-Ile or Tyr-Ser-Lys-Tyr (SEQ ID NO:3);

X10 is Met or an oxidatively stable amino acid;

X19 is Ala or Thr;

X20 is Arg, Lys, His or Ala;

P2 is Ile-Asn, Ile-Ala or Val-Gln;

P3 is a covalent bond, or is Ile, Ile-Thr or Ile-Thr-Asp;

R1 is H or an N-terminal blocking group;

R2 is OH or a C-terminal blocking group;

Y1 is one or two basic amino acids selected from the group Arg, Lys, and His;

Y2 is one or two basic amino acids selected from the group Arg, Lys, and His; and

m and n, independently, are 0 or 1;

wherein at least one of X1, X2, X3, X4, P1, X10, X19, X20, P2 and P3 is other than a wild type, mammalian GLP-2 residue; and

wherein when the peptide is administered in an effective dose to individuals experiencing one or more of parenteral nutrition, short bowel syndrome or intestinal failure, the GLP-2 analog has at least one of the following properties: prophylaxis of liver dysfunction, prophylaxis of kidney dysfunction, treatment of liver dysfunction, or treatment of kidney dysfunction.

GLP-2 peptide analogs may be analogs of full length GLP-2, i.e., GLP-2(1-33), and P3 is accordingly the sequence Ile-Thr-Asn. Alternatively, the GLP-2 analogs may be C-terminally truncated, to yield GLP-2(1-32) forms in which P3 is Ile-Thr, or GLP-2(1-31) forms in which P3 is Ile, or GLP-2(1-30) forms in which P3 is a covalent bond.

Furthermore, in certain embodiments, GLP-2 analogs may incorporate desired amino acid substitutions into a “background” which is an N-terminally or C-terminally modified form of a mammalian GLP-2 peptide. Such analogs are represented according to SEQ ID NO.:2 as those in which R1 constitutes an N-terminal blocking group, and/or when m is 1 then Y1 is one or two basic amino acids such as Arg or Lys; and/or R2 is a C-terminal blocking group; and/or when n is 1 then Y2 is independently, one or two basic amino acids such as Arg or Lys.

The “blocking groups” represented by R1 and R2 are chemical groups that are routinely used in the art of peptide chemistry to confer biochemical stability and resistance to digestion by exopeptidase. Suitable N-terminal protecting groups include, for example, O₁₋₅ alkanoyl groups such as acetyl. Also suitable as N-terminal protecting groups are amino acid analogs lacking the amino function. Suitable C-terminal protecting groups include groups which form ketones or amides at the carbon atom of the C-terminal carboxyl, or groups which form esters at the oxygen atom of the carboxyl. Ketone and ester-forming groups include alkyl groups, particularly branched or unbranched C₁₋₅ alkyl groups, e.g., methyl, ethyl and propyl groups, while amide-forming groups include amino functions such as primary amine, or alkylamino functions, e.g., mono-O₁₋₅ alkylamino and di-O₁₋₅ alkylamino groups such as methylamino, ethylamino, dimethylamino, diethylamino, methylethylamino and the like. Amino acid analogs are also suitable for protecting the C-terminal end of the present compounds, for example, decarboxylated amino acid analogs such as agmatine.

GLP-2 analogs can alternately be generated using standard techniques of peptide chemistry according to the guidance provided herein. Particularly preferred analogs for use in the invention are those based upon the sequence of human GLP-2 (SEQ ID NO: 4) wherein one or more amino acid residues are conservatively substituted for another amino acid residue, and wherein when the peptide is administered in an effective dose to individuals experiencing one or more of parenteral nutrition, short bowel syndrome or intestinal failure, the GLP-2 analog has at least one of the following properties: prophylaxis of liver dysfunction, prophylaxis of kidney dysfunction, treatment of liver dysfunction, or treatment of kidney dysfunction.

Conservative substitutions in any naturally occurring GLP-2, preferably the human GLP-2 sequence, are defined as exchanges of any member of the following five groups for another member of the same group:

I.
Ala, Ser, Thr, Pro, Gly
II.
Asn, Asp, Glu, Gln
III.
His, Arg, Lys
IV.
Met, Leu, Ile, Val, Cys
V.
Phe, Tyr, Trp.

In certain embodiments, GLP-2 analogs may be created by changing an amino acid residue in one mammalian GLP-2 to the corresponding amino acid in another mammalian GLP-2 peptide. Wild-type mammalian GLP-2 residues which occur at a specific position are determined by aligning the sequences of GLP-2's isolated from different mammalian species and comparing the sequence to the human sequence, reproduced below, for convenience (SEQ ID NO:3):

His-Ala-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp

The amino acid residues which, for purposes of this application, are known to vary at specific positions in wild type mammalian GLP-2s are the following (according to the notation of SEQ ID NO.:2): position X13, which may be Ile or Val; position X16, which may be Asn or Ser; position X19, which may be Alanine or Threonine; position X20, which may be Arg or Lys; position X27, which may be Ile or Leu; and position X28, which may be Gln or His.

GLP-2 analogs also include peptides with non-conservative substitutions of amino acids in any vertebrate GLP-2 sequence, provided that the non-conservative substitutions occur at amino acid positions known to vary in GLP-2 isolated from different species. Such non-conserved residue positions are readily determined by aligning all known vertebrate GLP-2 sequences. For example, Buhl et al., J. Biol. Chem., 1988, 263(18):8621, compared the sequences of human, porcine, rat, hamster, guinea pig, and bovine GLP-2's, and found that positions 13, 16, 19, 27 and 28 according to SEQ ID NO.:3 were non-conserved (position numbers refer to the analogous position in the human GLP-2 sequence). Nishi and Steiner, Mol. Endocrinol., 1990, 4:1192-8, found that an additional position corresponding to residue 20 of SEQ ID NO.:3 also varied in degu, a rodent species indigenous to South America. Thus, under this standard, the amino acid positions which vary in mammals and which preferably may be substituted with non-conservative residues are, according to the positions of SEQ ID NO.:3, positions 13, 16, 19, 20, 27, and 28. The additional amino acid residues which vary in vertebrates and which also may be substituted with non-conserved residues occur at positions 2, 5, 7, 8, 9, 10, 12, 17, 21, 22, 23, 24, 26, 29, 30, 31, 32, and 33 in SEQ ID NO.:3.

Alternatively, non-conservative substitutions may be made at any position by alanine-scanning provided that when the resulting peptide is administered in an effective dose to individuals experiencing one or more of parenteral nutrition, short bowel syndrome or intestinal failure, the GLP-2 analog has at least one of the following properties: prophylaxis of liver dysfunction, prophylaxis of kidney dysfunction, treatment of liver dysfunction, or treatment of kidney dysfunction. The technique of alanine scanning mutagenesis is described by Cunningham and Wells, Science, 1989, 244:1081, and incorporated herein by reference in its entirety. Since most GLP-2 sequences consist of only approximately 33 amino acids (and in human GLP-2 alanine already occurs at four positions), one of skill in the art could easily test an alanine analog at each remaining position for one or more of the effects of prophylaxis of liver dysfunction, prophylaxis of kidney dysfunction, treatment of liver dysfunction, or treatment of kidney dysfunction, as taught in Example 1 below.

One particular GLP-2 analog, teduglutide, is particularly useful because it is a dipeptidyl peptidase IV resistant GLP-2 analog with the peptide sequence:

(SEQ ID NO: 4)
His-Gly-Asp-Gly-Ser-Phe-Ser-Asp-Glu-Met-Asn-Thr-
Ile-Leu-Asp-Asn-Leu-Ala-Ala-Arg-Asp-Phe-Ile-Asn-
Trp-Leu-Ile-Gln-Thr-Lys-Ile-Thr-Asp

GLP-2 analogs are known in the art, and are further disclosed in U.S. Pat. No. 5,789,379, U.S. Pat. No. 5,834,428, U.S. Pat. No. 6,184,201, United States patent application publication number 20030162703 and United States patent application publication number 20060105954, all of which are hereby incorporated by reference in their entirety.

The term “diagnostic biomarker” refers herein to any measurable state of an organism, wherein measurement of that state is useful in diagnosing or determining the progression or regression of one or more diseases, or in determining the level of function of particular body systems or organs. Diagnostic biomarkers are well known and commonly used in the art, and one skilled in the art can readily choose a diagnostic biomarker to diagnose or follow the course of a particular disease, or to determine the level of function of particular body systems. Several examples of diagnostic biomarkers are given below for the purpose of illustration only.

Albumin is a diagnostic biomarker that can be used to assay liver function or dysfunction. Because albumin is synthesized in the liver, albumin levels may decrease in individuals experiencing liver dysfunction.

Bilirubin is a diagnostic biomarker frequently used to assess the function of the liver. Because bilirubin is cleared from the body through the liver, increased levels of bilirubin in an individual are associated with decreased liver function. The level of bilirubin is often measured by testing urine or blood using methods well known and commonly used in the art.

Gamma glutamyl transferase (GGT), alkaline phosphatase (ALP), alanine transaminase (ALT), and aspartate aminotransferase (AST) are also diagnostic biomarkers used to assess the function of the liver. These enzymes are highly concentrated in liver cells, and damage to liver cells releases these enzymes into the blood. Elevated levels of these enzymes, as determined by assaying an individual's blood using known methods, are indicative of liver damage and decreased liver function.

Examples of diagnostic biomarkers useful to assess kidney function include urea nitrogen, creatinine and glomerular filtration rate. Urea nitrogen and creatinine are both molecules that are cleared from the body in large part through the kidneys. Thus, higher levels of the urea nitrogen and creatinine diagnostic biomarkers in an individual's serum, as assayed using known methods, are associated with decreased liver function.

Glomerular filtration rate (GFR) is a measurement of the filtration capacity of an individual's kidneys. A higher GFR is associated with increased liver function. This diagnostic biomarker may be calculated through any method where the filtration capacity of the kidneys is measured. For example, inulin, a polysaccharide, is sometimes injected into an individual's plasma at a known concentration, and filtration of inulin into the individual's urine by the kidneys is monitored. Alternatively, GFR is often estimated by a formula well known in the art, which incorporates an individual's age, mass, and creatinine level in the serum.

Because many diagnostic biomarkers are associated with more than one disease or body system or organ function, often a panel of tests involving multiple diagnostic biomarkers will be ordered by a medical professional to provide a clearer assessment of a particular disease state or the level of function of a particular body system. For example, when liver function is in question, a liver function panel is often ordered wherein two or more of the diagnostic biomarkers useful in assessing liver function are ordered.

### Methods

The methods described herein are methods for treatment or prophylaxis of liver or kidney disease in individuals experiencing one or more of: parenteral nutrition, intestinal failure or short bowel syndrome. In these methods, GLP-2 or GLP-2 peptide analogs are administered to individuals. A researcher may determine whether a particular GLP-2 peptide or GLP-2 analog has a prophylactic effect against kidney or liver disease by administering the peptide or analog to individuals in danger of developing kidney or liver disease (e.g. individuals experiencing one or more of: parenteral nutrition, intestinal failure or short bowel syndrome.) The researcher would then determine, using diagnostic biomarkers, whether the individuals thus treated are less likely to develop liver or kidney dysfunction.

Likewise, a researcher can determine whether a particular GLP-2 peptide or analog may be used to treat kidney or liver disease by administering the peptide or analog to individuals who have kidney or liver disease. The researcher would then determine, using diagnostic biomarkers, whether the individuals thus treated show improvement in liver or kidney function.

The specific therapeutic regimens used to assess whether a molecule has a desired effect are well known in the art. A researcher faced with the task of determining whether a particular GLP-2 peptide or analog may be used for treatment or prophylaxis of kidney or liver disease would choose the appropriate regimen to make this determination.

Delivery methods and formulations useful for administering peptides to individuals are well known in the art, and a skilled person would be able to determine the suitability of any particular method of delivery of a peptide to an individual for particular circumstances. For the purposes of illustration only, the following examples of methods and formulations for administering peptides to individuals are provided.

Peptides may be administered to individuals orally, however, actions of the digestive system will generally greatly reduce the bioavailability of the peptide. In order to increase peptide oral bioavailability, peptides may be administered in formulations containing enzyme inhibitors, or the peptides may be administered as part of a micelle, nanoparticle or emulsion in order to protect the peptide from digestive activity.

Peptides may also be administered by means of an injection. The peptides may be injected subcutaneously, intramuscularly, or intravenously. Further disclosure regarding methods of administering peptides through injection is found in U.S. Pat. No. 5,952,301, which is hereby incorporated by reference in its entirety.

Peptides may further be administered by pulmonary delivery. A dry powder inhalation system may be used, wherein peptides are absorbed through the tissue of the lungs, allowing delivery without injection, while bypassing the potential reduction in bioavailability seen with oral administration (see Onoue et al., Expert Op. on Therapeutic Patents 18:429, 2008, which is hereby incorporated by reference).

A typical human dose of a GLP-2 peptide would be from about 10 μg/kg body weight/day to about 10 mg/kg/day, preferably from about 50 μg/kg/day to about 5 mg/kg/day, and most preferably from about 100 μg/kg/day to about 1 mg/kg/day. As the GLP-2 analogs can be from about 10 to even about 100 times more potent than GLP-2, a typical dose of such a GLP-2 analog may be lower, for example, from about 100 ng/kg body weight/day to 1 about mg/kg/day, preferably from about 1 μg/kg/day to about 500 μg/kg/day, and even more preferably from about 1 μg/kg/day to about 100 μg/kg/day.

In one aspect of the invention, a GLP-2 peptide, or a GLP-2 peptide analog may be used in a method to treat liver dysfunction. In one embodiment of a method to treat liver dysfunction, one or more of a GLP-2 peptide or a GLP-2 peptide analog are administered to an individual having impaired liver function in an amount sufficient to cause improvement of liver function, wherein the individual is experiencing one or more of the following: intestinal failure, short bowel syndrome or parenteral nutrition.

In several embodiments of the method to treat liver dysfunction of the invention, the GLP-2 peptide analog is teduglutide (SEQ ID NO.:4). In another embodiment, teduglutide is administered at a dose in the range of from about 0.001 mg/kg/day to about 10 mg/kg/day, from about 0.01 mg/kg/day to about 1 mg/kg/day, from about 0.05 mg/kg/day to about 0.2 mg/kg/day, from about 0.001 mg/kg/day to about 0.01 mg/kg/day, from about 0.01 mg/kg/day to about 0.1 mg/kg/day, from about 0.1 mg/kg/day to about 1 mg/kg/day, or from about 1 mg/kg/day to about 10 mg/kg/day.

In some embodiments, improvement in liver function may be observed in a time frame of less than one, one, two, three, four, five, six, seven, eight, nine, ten, eleven, twelve, or more than twelve weeks after the beginning of administration of one or more of GLP-2 peptide or GLP-2 peptide analog to the individual with liver dysfunction.

In another embodiment of a method to treat liver dysfunction, increased liver function is determined through the use of at least one diagnostic biomarker. In some such methods, the diagnostic biomarker used is selected from the group consisting of: bilirubin, alanine transaminase, aspartate aminotransferase, and alkaline phosphatase. In another embodiment, at least one of the tested biomarkers decreases by at least 1, 2, 3, 4, 5, 10, 15, 20, 25, 30, 35, 40, 45, 50, 60, 70, 80, 90, 100, 200, 500, or 1000 percent after treatment with GLP-2 or GLP-2 analog.

In another aspect of the invention, GLP-2 peptide, or a GLP-2 peptide analog may be used in a method for prophylaxis against liver dysfunction. In one embodiment of a method for prophylaxis against liver dysfunction, one or more of a GLP-2 peptide or a GLP-2 peptide analog are administered to an individual in an amount sufficient for prophylaxis against liver dysfunction, wherein the individual is experiencing one or more of the following: intestinal failure, short bowel syndrome or parenteral nutrition.

In another embodiment of a method for prophylaxis against liver dysfunction, the GLP-2 peptide analog is teduglutide (SEQ ID NO:4). In another embodiment, teduglutide is administered at a dose in the range of from about 0.001 mg/kg/day to about 10 mg/kg/day, from about 0.01 mg/kg/day to about 1 mg/kg/day, from about 0.05 mg/kg/day to about 0.2 mg/kg/day, from about 0.001 mg/kg/day to about 0.01 mg/kg/day, from about 0.01 mg/kg/day to about 0.1 mg/kg/day, from about 0.1 mg/kg/day to about 1 mg/kg/day, or from about 1 mg/kg/day to about 10 mg/kg/day.

In another embodiment, prophylaxis against liver dysfunction occurs in a time frame of less than one, one, two, three, four, five, six, seven, eight, nine, ten, eleven, twelve, or more than twelve weeks after the beginning of administration of one or more of GLP-2 peptide or GLP-2 peptide analog to the individual.

In another embodiment of a method for prophylaxis against liver dysfunction, liver function is monitored through the use of diagnostic biomarkers. In another embodiment, the diagnostic biomarkers used are selected from the group consisting of: bilirubin, alanine transaminase, aspartate aminotransferase, and alkaline phosphatase.

In another embodiment, at least one of the tested biomarkers increases, if at all, by less than 0.1, 0.5, 1, 2, 3, 4, 5, 6, 7, 8, 9, 10, 15, 20, 25, 30, 35, 40, 45, 50, 60, 70, 80, 90 or 100 percent after treatment with GLP-2 or GLP-2 analog.

In another aspect of the invention, GLP-2 peptide, or a GLP-2 peptide analog may be used in a method to treat kidney dysfunction. In one embodiment of a method to treat kidney dysfunction, one or more of a GLP-2 peptide or a GLP-2 peptide analog are administered to an individual having impaired kidney function in an amount sufficient to cause improvement of liver function, wherein the individual is experiencing one or more of the following: intestinal failure, short bowel syndrome or parenteral nutrition.

In another embodiment of a method to treat kidney dysfunction, the GLP-2 peptide analog is teduglutide SEQ ID NO.:4). In another embodiment, teduglutide is administered at a dose in the range of from about 0.001 mg/kg/day to about 10 mg/kg/day, from about 0.01 mg/kg/day to about 1 mg/kg/day, from about 0.05 mg/kg/day to about 0.2 mg/kg/day, from about 0.001 mg/kg/day to about 0.01 mg/kg/day, from about 0.01 mg/kg/day to about 0.1 mg/kg/day, from about 0.1 mg/kg/day to about 1 mg/kg/day, or from about 1 mg/kg/day to about 10 mg/kg/day.

In another embodiment, improvement in kidney function occurs in a time frame of less than one, one, two, three, four, five, six, seven, eight, nine, ten, eleven, twelve, or more than twelve weeks after the beginning of administration of one or more of GLP-2 peptide or GLP-2 peptide analog to the individual with kidney dysfunction.

In another embodiment of a method to treat kidney dysfunction, increased kidney function is determined through the use of diagnostic biomarkers. In another embodiment, the diagnostic biomarkers used are selected from the group consisting of: urea nitrogen, creatinine and glomerular filtration rate. In another embodiment, one or more of the individual level of urea nitrogen, or creatinine, if selected, decreases at least 5, about 5, 10, 20, 50, 100, 200, 500, 1000 or more than 1000 percent after treatment with GLP-2 or GLP-2 analog or the level of glomerular filtration rate, if selected, increases by at least 1, 2, 3, 4, 5, 10, 15, 20, 25, 30, 35, 40, 45, 50, 60, 70, 80, 90, 100, 200, 500, or 1000 percent after treatment with GLP-2 or GLP-2 analog.

In another aspect of the invention, a GLP-2 peptide, or a GLP-2 peptide analog may be used in a method for prophylaxis against kidney dysfunction. In one embodiment of a method for prophylaxis against kidney dysfunction, one or more of a GLP-2 peptide or a GLP-2 peptide analog are administered to an individual in an amount sufficient for prophylaxis against liver dysfunction, wherein the individual is experiencing one or more of the following: intestinal failure, short bowel syndrome or parenteral nutrition.

In another embodiment of a method for prophylaxis against kidney dysfunction, the GLP-2 peptide analog is teduglutide (SEQ ID NO.:4). In another embodiment, teduglutide is administered at a dose in the range of from about 0.001 mg/kg/day to about 10 mg/kg/day, from about 0.01 mg/kg/day to about 1 mg/kg/day, from about 0.05 mg/kg/day to about 0.2 mg/kg/day, from about 0.001 mg/kg/day to about 0.01 mg/kg/day, from about 0.01 mg/kg/day to about 0.1 mg/kg/day, from about 0.1 mg/kg/day to about 1 mg/kg/day, or from about 1 mg/kg/day to about 10 mg/kg/day.

In another embodiment, prophylaxis of kidney dysfunction occurs in a time frame of less than one, one, two, three, four, five, six, seven, eight, nine, ten, eleven, twelve, or more than twelve weeks after the beginning of administration of one or more of GLP-2 peptide or GLP-2 peptide analog to the individual.

In another embodiment of a method for prophylaxis against kidney dysfunction, kidney function is monitored through the use of diagnostic biomarkers. In another embodiment, the diagnostic biomarkers used are selected from the group consisting of: urea nitrogen, creatinine and glomerular filtration rate. In another embodiment, one or more of the individual level of urea nitrogen, or creatinine, if selected, increases, if at all, by less than 0.1, 0.5, 1, 2, 3, 4 or 5 percent after treatment with GLP-2 or GLP-2 analog or the level of glomerular filtration rate, if selected, decreases, if at all, by less than 0.1, 0.5, 1, 2, 3, 4, 5, 6, 7, 8, 9, 10, 15, 20, 25, 30, 35, 40, 45, 50, 60, 70, 80, 90 or 100 percent after treatment with GLP-2 or GLP-2 analog.

The specific examples included hereafter are for illustrative purposes only and are not to be considered as limiting to this disclosure.

### Example 1

Human patients with short bowel syndrome were divided into three groups: a control group (16 total individuals) to receive placebo, an experimental group (35 total individuals) to receive teduglutide at 0.05 mg/kg/day throughout the study, and an experimental group (32 total individuals) to receive teduglutide at 0.1 mg/kg/day throughout the study. Patients were treated by subcutaneous injection of placebo or the appropriate dose of teduglutide. As individuals with short bowel syndrome are likely to experience associated liver and kidney disease, known biomarkers of liver and kidney function were monitored every four weeks, for 24 weeks. The liver biomarkers tested included total bilirubin, ALT and AST and ALP. The kidney biomarkers tested included urea nitrogen, creatinine and GFR. Before administration of placebo or teduglutide, diagnostic biomarker levels were tested to establish a baseline for each individual.

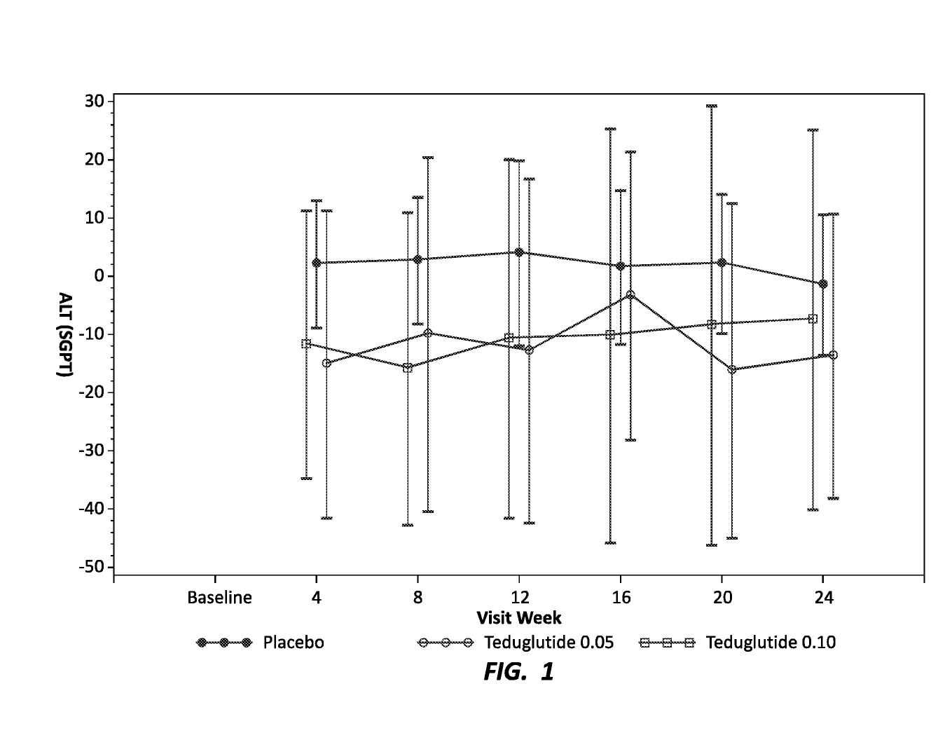

To visualize the effect of GLP-2 on the levels of the diagnostic biomarkers, the average change from baseline was calculated for the placebo and teduglutide treated groups for each visit after a baseline was established. The average change from baseline of each group is depicted in FIGS. 1-7. The numerical figures for each diaanostic biomarker at each time point are depicted in Table 1.

TABLE 1
Selected Lab Tests Results

| | | Placebo | 0.05 | 0.10 | p-value | p-value | p-value |
|---|---|---|---|---|---|---|---|
| Lab Tests | Visits | (N = 16) | (N = 35) | (N = 32) | Placebo vs. 0.05 | Placebo vs. 0.10 | 0.05 vs. 0.10 |
| ALT (SGPT) | | | | | | | |
| N | 2.0 Baseline | 15 | 34 | 32 | | | |
| Mean (SD) | | 35.8 (19.06) | 45.9 (33.41) | 50.2 (35.70) | 0.2817 | 0.1519 | 0.6171 |
| Median | | 32.0 | 32.0 | 43.0 | | | |
| Range | | 16, 69 | 14, 137 | 12, 174 | | | |
| N | 3.0 DW4 | 16 | 31 | 32 | | | |
| Mean (SD) | | 38.0 (22.45) | 30.1 (14.08) | 38.6 (23.30) | 0.1453 | 0.9332 | 0.0860 |
| Median | | 31.0 | 27.0 | 30.0 | | | |
| Range | | 14, 80 | 11, 68 | 10, 105 | | | |
| N | 4.0 DW8 | 16 | 30 | 30 | | | |
| Mean (SD) | | 43.2 (28.57) | 37.3 (23.71) | 34.1 (19.74) | 0.4618 | 0.2111 | 0.5682 |
| Median | | 32.0 | 31.5 | 28.5 | | | |
| Range | | 15, 111 | 12, 107 | 9, 93 | | | |
| N | 5.0 DW12 | 16 | 28 | 29 | | | |
| Mean (SD) | | 38.3 (24.61) | 34.6 (24.30) | 40.4 (22.11) | 0.6307 | 0.7744 | 0.3521 |
| Median | | 31.0 | 26.0 | 35.0 | | | |
| Range | | 13, 92 | 11, 125 | 11, 102 | | | |
| N | 6.0 DW16 | 15 | 29 | 30 | | | |
| Mean (SD) | | 37.5 (21.79) | 39.5 (39.44) | 39.7 (27.97) | 0.8577 | 0.7943 | 0.9836 |
| Median | | 34.0 | 28.0 | 30.0 | | | |
| Range | | 14, 81 | 10, 219 | 8, 137 | | | |
| N | 7.0 DW20 | 16 | 28 | 29 | | | |
| Mean (SD) | | 36.4 (23.76) | 29.7 (13.35) | 41.3 (29.36) | 0.2344 | 0.5755 | 0.0624 |
| Median | | 31.5 | 26.0 | 29.0 | | | |
| Range | | 11, 93 | 12, 61 | 9, 104 | | | |
| N | 8.0 DW 24 | 16 | 27 | 29 | | | |
| Mean (SD) | | 33.4 (17.87) | 31.7 (26.63) | 42.2 (27.28) | 0.8211 | 0.2499 | 0.1484 |
| Median | | 27.5 | 20.0 | 37.0 | | | |
| Range | | 14, 66 | 8, 134 | 9, 120 | | | |
| ALT (SGPT) | | | | | | | |
| Change from | | | | | | | |
| Baseline | | | | | | | |
| N | 3.0 DW4 | 15 | 30 | 32 | | | |
| Mean (SD) | | 2.3 (10.92) | −14.9 (26.39) | −11.6 (22.98) | 0.0202 | 0.0325 | 0.5931 |
| Median | | 2.0 | −5.0 | −6.5 | | | |
| Range | | −20, 32 | −100, 13 | −69, 29 | | | |
| N | 4.0 DW8 | 15 | 29 | 30 | | | |
| Mean (SD) | | 2.9 (10.88) | −9.8 (30.38) | −15.7 (26.87) | 0.1273 | 0.0140 | 0.4317 |
| Median | | 3.0 | −4.0 | −8.0 | | | |
| Range | | −12, 30 | −103, 49 | −126, 13 | | | |
| N | 5.0 DW12 | 15 | 27 | 29 | | | |
| Mean (SD) | | 4.1 (15.87) | −12.7 (29.55) | −10.6 (30.82) | 0.0483 | 0.0920 | 0.7945 |
| Median | | 1.0 | −10.0 | −11.0 | | | |
| Range | | −22, 41 | −83, 70 | −107, 61 | | | |
| N | 6.0 DW16 | 15 | 28 | 30 | | | |
| Mean (SD) | | 1.7 (13.23) | −3.2 (24.76) | −10.0 (35.59) | 0.4798 | 0.2244 | 0.4013 |
| Median | | −1.0 | −2.5 | −10.0 | | | |
| Range | | −20, 30 | −56, 82 | −124, 87 | | | |
| N | 7.0 DW20 | 15 | 27 | 29 | | | |
| Mean (SD) | | 2.3 (11.96) | −16.0 (28.73) | −8.2 (37.70) | 0.0235 | 0.2979 | 0.3906 |
| Median | | 1.0 | −5.0 | −9.0 | | | |
| Range | | −21, 26 | −107, 12 | −130, 77 | | | |
| N | 8.0 DW 24 | 15 | 26 | 29 | | | |
| Mean (SD) | | −1.3 (12.07) | −13.5 (24.40) | −7.3 (32.62) | 0.0785 | 0.5010 | 0.4281 |
| Median | | −1.0 | −5.0 | −4.0 | | | |
| Range | | −25, 27 | −70, 45 | −133, 60 | | | |
| AST (SGOT) | | | | | | | |
| N | 2.0 Baseline | 15 | 34 | 32 | | | |
| Mean (SD) | | 33.6 (13.27) | 37.9 (21.78) | 41.1 (21.79) | 0.4851 | 0.2249 | 0.5478 |
| Median | | 28.0 | 28.5 | 36.5 | | | |
| Range | | 20, 55 | 12, 106 | 17, 98 | | | |
| N | 3.0 DW4 | 16 | 29 | 31 | | | |
| Mean (SD) | | 33.7 (14.08) | 29.1 (19.23) | 33.5 (17.00) | 0.4077 | 0.9777 | 0.3460 |
| Median | | 33.0 | 27.0 | 28.0 | | | |
| Range | | 14, 59 | 11, 121 | 16, 96 | | | |
| N | 4.0 DW8 | 16 | 30 | 30 | | | |
| Mean (SD) | | 35.6 (17.71) | 33.5 (18.56) | 29.3 (12.52) | 0.7046 | 0.1636 | 0.3084 |
| Median | | 28.0 | 28.0 | 28.0 | | | |
| Range | | 17, 69 | 14, 88 | 12, 63 | | | |
| N | 5.0 DW12 | 16 | 28 | 28 | | | |
| Mean (SD) | | 32.9 (15.73) | 29.2 (15.48) | 36.4 (20.41) | 0.4531 | 0.5512 | 0.1401 |
| Median | | 25.0 | 26.5 | 31.5 | | | |
| Range | | 18, 72 | 15, 95 | 15, 118 | | | |
| N | 6.0 DW16 | 15 | 29 | 29 | | | |
| Mean (SD) | | 33.5 (15.64) | 29.3 (12.05) | 33.0 (16.71) | 0.3374 | 0.9237 | 0.3481 |
| Median | | 29.0 | 25.0 | 30.0 | | | |
| Range | | 18, 70 | 14, 74 | 14, 70 | | | |
| N | 7.0 DW20 | 16 | 28 | 28 | | | |
| Mean (SD) | | 34.3 (17.59) | 26.1 (7.02) | 34.9 (19.82) | 0.0344 | 0.9277 | 0.0326 |
| Median | | 27.5 | 25.0 | 30.0 | | | |
| Range | | 16, 74 | 14, 44 | 14, 105 | | | |
| N | 8.0 DW 24 | 16 | 27 | 29 | | | |
| Mean (SD) | | 29.8 (11.89) | 27.7 (10.23) | 35.3 (18.69) | 0.5420 | 0.2915 | 0.0658 |
| Median | | 27.0 | 25.0 | 32.0 | | | |
| Range | | 13, 56 | 12, 55 | 11, 96 | | | |
| AST (SGOT) | | | | | | | |
| Change from | | | | | | | |
| Baseline | | | | | | | |
| N | 3.0 DW4 | 15 | 28 | 31 | | | |
| Mean (SD) | | −0.7 (7.85) | −5.5 (24.09) | −7.3 (15.21) | 0.4624 | 0.1265 | 0.7362 |
| Median | | −2.0 | −4.0 | −5.0 | | | |
| Range | | −11, 19 | −50, 96 | −44, 18 | | | |
| N | 4.0 DW8 | 15 | 29 | 30 | | | |
| Mean (SD) | | 0.1 (10.20) | −3.2 (19.88) | −10.3 (16.56) | 0.5463 | 0.0309 | 0.1414 |
| Median | | 2.0 | −3.0 | −5.0 | | | |
| Range | | −26, 16 | −45, 63 | −62, 11 | | | |
| N | 5.0 DW12 | 15 | 27 | 28 | | | |
| Mean (SD) | | −0.2 (12.58) | −5.9 (19.28) | −3.3 (21.39) | 0.3146 | 0.6157 | 0.6379 |
| Median | | −4.0 | −8.0 | −3.5 | | | |
| Range | | −23, 25 | −42, 70 | −43, 68 | | | |
| N | 6.0 DW16 | 15 | 28 | 29 | | | |
| Mean (SD) | | −0.1 (8.29) | −4.3 (11.75) | −6.6 (22.80) | 0.2280 | 0.2973 | 0.6411 |
| Median | | −3.0 | −1.5 | −4.0 | | | |
| Range | | −11, 17 | −39, 18 | −56, 49 | | | |
| N | 7.0 DW20 | 15 | 27 | 28 | | | |
| Mean (SD) | | 1.5 (11.13) | −8.0 (14.96) | −4.9 (24.96) | 0.0366 | 0.3480 | 0.5794 |
| Median | | −4.0 | −4.0 | −2.5 | | | |
| Range | | −15, 26 | −61, 6 | −62, 55 | | | |
| N | 8.0 DW 24 | 15 | 26 | 29 | | | |
| Mean (SD) | | −3.8 (12.49) | −6.8 (14.29) | −4.8 (23.34) | 0.5069 | 0.8748 | 0.7153 |
| Median | | 0.0 | −4.5 | −3.0 | | | |
| Range | | −42, 9 | −45, 22 | −66, 46 | | | |
| CREATININE | | | | | | | |
| (RATE BLANKED) | | | | | | | |
| N | 2.0 Baseline | 16 | 34 | 32 | | | |
| Mean (SD) | | 84.9 (27.44) | 80.8 (33.61) | 82.8 (19.70) | 0.6716 | 0.7627 | 0.7686 |
| Median | | 77.5 | 67.5 | 80.0 | | | |
| Range | | 44, 141 | 37, 177 | 49, 115 | | | |
| N | 3.0 DW4 | 16 | 31 | 32 | | | |
| Mean (SD) | | 88.3 (26.81) | 84.8 (30.01) | 81.0 (21.62) | 0.6962 | 0.3136 | 0.5647 |
| Median | | 87.5 | 80.0 | 83.0 | | | |
| Range | | 44, 135 | 42, 152 | 44, 128 | | | |
| N | 4.0 DW8 | 16 | 30 | 31 | | | |
| Mean (SD) | | 87.6 (32.96) | 81.9 (27.34) | 84.2 (21.41) | 0.5392 | 0.6710 | 0.7239 |
| Median | | 79.0 | 71.0 | 86.0 | | | |
| Range | | 44, 177 | 40, 155 | 53, 124 | | | |
| N | 5.0 DW12 | 16 | 28 | 30 | | | |
| Mean (SD) | | 92.1 (34.00) | 86.8 (29.41) | 86.4 (25.37) | 0.5846 | 0.5189 | 0.9577 |
| Median | | 80.0 | 80.0 | 84.5 | | | |
| Range | | 53, 177 | 44, 169 | 53, 143 | | | |
| N | 6.0 DW16 | 15 | 29 | 30 | | | |
| Mean (SD) | | 85.5 (30.37) | 82.1 (29.95) | 84.2 (21.85) | 0.7243 | 0.8699 | 0.7591 |
| Median | | 78.0 | 79.0 | 87.5 | | | |
| Range | | 44, 142 | 35, 155 | 44, 124 | | | |
| N | 7.0 DW20 | 16 | 28 | 29 | | | |
| Mean (SD) | | 89.1 (28.96) | 85.6 (28.77) | 86.6 (20.44) | 0.6962 | 0.7367 | 0.8741 |
| Median | | 83.5 | 77.5 | 88.0 | | | |
| Range | | 53, 144 | 42, 150 | 57, 133 | | | |
| N | 8.0 DW 24 | 16 | 27 | 29 | | | |
| Mean (SD) | | 88.2 (25.60) | 82.8 (31.57) | 82.0 (22.35) | 0.5646 | 0.4058 | 0.9189 |
| Median | | 80.0 | 80.0 | 81.0 | | | |
| Range | | 47, 133 | 35, 155 | 44, 130 | | | |
| CREATININE | | | | | | | |
| (RATE BLANKED) | | | | | | | |
| Change from | | | | | | | |
| Baseline | | | | | | | |
| N | 3.0 DW4 | 16 | 30 | 32 | | | |
| Mean (SD) | | 3.4 (11.35) | 1.2 (12.01) | −1.8 (10.49) | 0.5483 | 0.1207 | 0.2966 |
| Median | | 2.5 | 1.5 | −0.5 | | | |
| Range | | −26, 18 | −38, 18 | −23, 21 | | | |
| N | 4.0 DW8 | 16 | 29 | 31 | | | |
| Mean (SD) | | 2.6 (24.64) | −1.1 (15.51) | 1.2 (10.40) | 0.5360 | 0.7851 | 0.4946 |
| Median | | 0.0 | 0.0 | 0.0 | | | |
| Range | | −44, 80 | −43, 27 | −27, 22 | | | |
| N | 5.0 DW12 | 16 | 27 | 30 | | | |
| Mean (SD) | | 7.2 (21.95) | 1.8 (16.71) | 2.8 (12.77) | 0.3703 | 0.3946 | 0.8024 |
| Median | | 3.5 | 8.0 | 6.5 | | | |
| Range | | −17, 80 | −60, 19 | −36, 36 | | | |
| N | 6.0 DW16 | 15 | 28 | 30 | | | |
| Mean (SD) | | 4.3 (14.49) | −1.6 (13.06) | 0.6 (11.86) | 0.1860 | 0.3693 | 0.5097 |
| Median | | 4.0 | 0.0 | 0.0 | | | |
| Range | | −18, 44 | −47, 21 | −26, 27 | | | |
| N | 7.0 DW20 | 16 | 27 | 29 | | | |
| Mean (SD) | | 4.2 (9.11) | 0.7 (13.11) | 2.6 (12.05) | 0.3501 | 0.6531 | 0.5636 |
| Median | | 6.5 | 0.0 | 0.0 | | | |
| Range | | −13, 18 | −27, 18 | −36, 21 | | | |
| N | 8.0 DW 24 | 16 | 26 | 29 | | | |
| Mean (SD) | | 3.3 (14.89) | −2.9 (14.03) | −2.0 (13.18) | 0.1864 | 0.2315 | 0.8032 |
| Median | | 3.5 | −0.5 | 0.0 | | | |
| Range | | −26, 35 | −49, 20 | −37, 23 | | | |
| GFR | | | | | | | |
| N | 2.0 Baseline | 16 | 34 | 32 | | | |
| Mean (SD) | | 80.8 (29.31) | 84.9 (31.25) | 76.7 (26.52) | 0.6584 | 0.6333 | 0.2577 |
| Median | | 77.4 | 78.7 | 74.4 | | | |
| Range | | 29, 133 | 31, 194 | 34, 159 | | | |
| N | 3.0 DW4 | 16 | 31 | 32 | | | |
| Mean (SD) | | 77.1 (29.04) | 86.1 (36.99) | 80.3 (25.98) | 0.3989 | 0.6987 | 0.4704 |
| Median | | 72.2 | 77.0 | 75.6 | | | |
| Range | | 31, 130 | 40, 221 | 31, 137 | | | |
| N | 4.0 DW8 | 16 | 29 | 31 | | | |
| Mean (SD) | | 80.6 (37.65) | 89.1 (39.16) | 77.9 (25.63) | 0.4874 | 0.7684 | 0.1930 |
| Median | | 76.0 | 81.6 | 76.9 | | | |
| Range | | 32, 191 | 38, 233 | 37, 138 | | | |
| N | 5.0 DW12 | 16 | 27 | 30 | | | |
| Mean (SD) | | 76.8 (32.54) | 85.7 (39.48) | 77.3 (25.57) | 0.4503 | 0.9527 | 0.3399 |
| Median | | 74.2 | 76.5 | 68.7 | | | |
| Range | | 29, 161 | 35, 218 | 34, 126 | | | |
| N | 6.0 DW16 | 14 | 29 | 30 | | | |
| Mean (SD) | | 84.0 (38.95) | 89.8 (38.02) | 78.3 (23.12) | 0.6440 | 0.5493 | 0.1655 |
| Median | | 79.0 | 84.1 | 77.5 | | | |
| Range | | 30, 194 | 43, 222 | 32, 125 | | | |
| N | 7.0 DW20 | 16 | 28 | 29 | | | |
| Mean (SD) | | 78.0 (34.24) | 83.2 (32.77) | 77.3 (26.45) | 0.6183 | 0.9402 | 0.4547 |
| Median | | 73.5 | 74.0 | 76.1 | | | |
| Range | | 28, 163 | 37, 191 | 35, 147 | | | |
| N | 8.0 DW 24 | 16 | 27 | 29 | | | |
| Mean (SD) | | 76.3 (24.48) | 89.7 (41.28) | 82.4 (27.05) | 0.2468 | 0.4593 | 0.4356 |
| Median | | 77.4 | 78.1 | 81.1 | | | |
| Range | | 29, 133 | 43, 223 | 40, 130 | | | |
| GFR Change from | | | | | | | |
| Baseline | | | | | | | |
| N | 3.0 DW4 | 16 | 30 | 32 | | | |
| Mean (SD) | | −3.7 (7.92) | −1.4 (12.80) | 3.6 (11.81) | 0.5087 | 0.0316 | 0.1212 |
| Median | | −2.3 | −0.6 | 3.1 | | | |
| Range | | −22, 8 | −52, 21 | −41, 26 | | | |
| N | 4.0 DW8 | 16 | 28 | 31 | | | |
| Mean (SD) | | −0.1 (18.84) | −0.1 (19.13) | 0.6 (10.87) | 0.9992 | 0.8710 | 0.8617 |
| Median | | −0.4 | 1.1 | 0.2 | | | |
| Range | | −26, 58 | −73, 38 | −23, 32 | | | |
| N | 5.0 DW12 | 16 | 26 | 30 | | | |
| Mean (SD) | | −3.9 (13.27) | −3.1 (12.18) | 0.2 (15.54) | 0.8358 | 0.3743 | 0.3887 |
| Median | | −2.3 | −5.0 | −0.5 | | | |
| Range | | −26, 28 | −21, 27 | −47, 50 | | | |
| N | 6.0 DW16 | 14 | 28 | 30 | | | |
| Mean (SD) | | −1.1 (20.00) | 0.8 (12.08) | 1.1 (13.73) | 0.7123 | 0.6722 | 0.9161 |
| Median | | −4.2 | 0.9 | 1.1 | | | |
| Range | | −20, 61 | −21, 31 | −34, 32 | | | |
| N | 7.0 DW20 | 16 | 27 | 29 | | | |
| Mean (SD) | | −2.8 (11.98) | −2.0 (16.60) | −0.6 (16.16) | 0.8765 | 0.6345 | 0.7386 |
| Median | | −6.1 | 0.9 | 1.5 | | | |
| Range | | −19, 30 | −52, 29 | −44, 51 | | | |
| N | 8.0 DW 24 | 16 | 26 | 29 | | | |
| Mean (SD) | | −4.4 (17.25) | 3.7 (12.81) | 4.6 (16.73) | 0.0884 | 0.0950 | 0.8311 |
| Median | | −0.0 | 1.7 | 2.3 | | | |
| Range | | −51, 22 | −13, 40 | −29, 53 | | | |
| ALKALINE | | | | | | | |
| PHOSPHATASE | | | | | | | |
| N | 2.0 Baseline | 16 | 34 | 32 | | | |
| Mean (SD) | | 162.1 (118.63) | 183.6 (95.32) | 158.9 (78.50) | 0.4952 | 0.9118 | 0.2567 |
| Median | | 116.0 | 163.0 | 143.0 | | | |
| Range | | 53, 502 | 54, 444 | 58, 376 | | | |
| N | 3.0 DW4 | 16 | 31 | 32 | | | |
| Mean (SD) | | 156.9 (91.52) | 159.5 (90.91) | 147.5 (74.98) | 0.9263 | 0.7072 | 0.5707 |
| Median | | 143.5 | 139.0 | 124.0 | | | |
| Range | | 53, 361 | 47, 463 | 64, 362 | | | |
| N | 4.0 DW8 | 16 | 30 | 31 | | | |
| Mean (SD) | | 173.6 (149.49) | 152.2 (84.05) | 139.2 (75.84) | 0.5359 | 0.2988 | 0.5290 |
| Median | | 127.5 | 134.0 | 114.0 | | | |
| Range | | 53, 602 | 40, 410 | 52, 314 | | | |
| N | 5.0 DW12 | 16 | 28 | 30 | | | |
| Mean (SD) | | 156.6 (97.09) | 143.5 (73.92) | 145.9 (83.34) | 0.6170 | 0.6983 | 0.9069 |
| Median | | 143.0 | 130.5 | 115.0 | | | |
| Range | | 45, 377 | 38, 376 | 42, 405 | | | |
| N | 6.0 DW16 | 15 | 29 | 30 | | | |
| Mean (SD) | | 149.5 (87.48) | 168.2 (128.08) | 137.7 (81.33) | 0.6152 | 0.6567 | 0.2777 |
| Median | | 119.0 | 134.0 | 108.5 | | | |
| Range | | 52, 386 | 52, 667 | 49, 400 | | | |
| N | 7.0 DW20 | 16 | 28 | 29 | | | |
| Mean (SD) | | 145.7 (80.23) | 144.2 (76.09) | 137.1 (82.59) | 0.9508 | 0.7367 | 0.7369 |
| Median | | 116.5 | 136.5 | 107.0 | | | |
| Range | | 65, 336 | 38, 363 | 52, 389 | | | |
| N | 8.0 DW 24 | 16 | 26 | 29 | | | |
| Mean (SD) | | 138.8 (84.49) | 169.6 (204.22) | 136.7 (85.50) | 0.5704 | 0.9374 | 0.4304 |
| Median | | 116.0 | 131.0 | 105.0 | | | |
| Range | | 69, 400 | 38, 1139 | 41, 430 | | | |
| ALKALINE | | | | | | | |
| PHOSPHATASE | | | | | | | |
| Change from | | | | | | | |
| Baseline | | | | | | | |
| N | 3.0 DW4 | 16 | 30 | 32 | | | |
| Mean (SD) | | −5.2 (44.57) | −24.8 (45.66) | −11.3 (32.19) | 0.1696 | 0.5863 | 0.1838 |
| Median | | −2.0 | −16.0 | −10.5 | | | |
| Range | | −158, 44 | −194, 51 | −86, 65 | | | |
| N | 4.0 DW8 | 16 | 29 | 31 | | | |
| Mean (SD) | | 11.5 (42.54) | −24.0 (64.47) | −21.2 (41.41) | 0.0547 | 0.0146 | 0.8369 |
| Median | | 2.0 | −20.0 | −20.0 | | | |
| Range | | −87, 100 | −209, 149 | −110, 118 | | | |
| N | 5.0 DW12 | 16 | 27 | 30 | | | |
| Mean (SD) | | −5.5 (39.84) | −39.4 (48.95) | −13.1 (55.43) | 0.0239 | 0.6319 | 0.0637 |
| Median | | −0.5 | −24.0 | −16.5 | | | |
| Range | | −133, 43 | −204, 21 | −129, 146 | | | |
| N | 6.0 DW16 | 15 | 28 | 30 | | | |
| Mean (SD) | | 10.1 (22.36) | −12.3 (69.90) | −21.3 (59.79) | 0.2363 | 0.0571 | 0.6004 |
| Median | | 4.0 | −20.5 | −20.5 | | | |
| Range | | −19, 68 | −127, 266 | −141, 196 | | | |
| N | 7.0 DW20 | 16 | 27 | 29 | | | |
| Mean (SD) | | −16.4 (103.44) | −36.9 (49.89) | −18.5 (63.67) | 0.3863 | 0.9328 | 0.2370 |
| Median | | 6.5 | −25.0 | −23.0 | | | |
| Range | | −396, 59 | −171, 59 | −176, 194 | | | |
| N | 8.0 DW 24 | 16 | 25 | 29 | | | |
| Mean (SD) | | −23.3 (86.39) | −11.6 (166.86) | −18.9 (67.08) | 0.7973 | 0.8497 | 0.8296 |
| Median | | −1.0 | −29.0 | −21.0 | | | |
| Range | | −325, 48 | −200, 738 | −171, 235 | | | |
| TOTAL | | | | | | | |
| BILIRUBIN | | | | | | | |
| N | 2.0 Baseline | 16 | 32 | 32 | | | |
| Mean (SD) | | 10.8 (8.01) | 11.2 (9.10) | 12.8 (21.85) | 0.8711 | 0.7258 | 0.7101 |
| Median | | 9.5 | 9.0 | 9.0 | | | |
| Range | | 3, 38 | 3, 44 | 3, 130 | | | |
| N | 3.0 DW4 | 16 | 28 | 31 | | | |
| Mean (SD) | | 11.1 (8.10) | 10.4 (9.48) | 9.0 (5.65) | 0.8041 | 0.3210 | 0.5124 |
| Median | | 8.5 | 7.0 | 7.0 | | | |
| Range | | 4, 36 | 3, 46 | 3, 34 | | | |
| N | 4.0 DW8 | 16 | 27 | 29 | | | |
| Mean (SD) | | 13.4 (17.06) | 10.8 (8.86) | 9.4 (9.80) | 0.5139 | 0.3305 | 0.5975 |
| Median | | 8.0 | 8.0 | 7.0 | | | |
| Range | | 3, 75 | 3, 36 | 3, 56 | | | |
| N | 5.0 DW12 | 16 | 25 | 29 | | | |
| Mean (SD) | | 11.5 (9.47) | 9.4 (6.65) | 10.3 (11.39) | 0.4177 | 0.7319 | 0.7284 |
| Median | | 9.5 | 7.0 | 7.0 | | | |
| Range | | 3, 39 | 3, 26 | 3, 65 | | | |
| N | 6.0 DW16 | 15 | 27 | 28 | | | |
| Mean (SD) | | 11.5 (7.50) | 10.0 (8.90) | 9.8 (9.14) | 0.5831 | 0.5368 | 0.9306 |
| Median | | 9.0 | 7.0 | 7.5 | | | |
| Range | | 4, 31 | 3, 43 | 4, 53 | | | |
| N | 7.0 DW20 | 16 | 26 | 29 | | | |
| Mean (SD) | | 10.1 (5.35) | 9.7 (6.78) | 10.4 (10.13) | 0.8444 | 0.9162 | 0.7727 |
| Median | | 8.5 | 9.0 | 7.0 | | | |
| Range | | 4, 22 | 3, 31 | 3, 56 | | | |
| N | 8.0 DW 24 | 16 | 23 | 27 | | | |
| Mean (SD) | | 10.7 (5.31) | 10.0 (6.28) | 11.3 (12.28) | 0.7060 | 0.8522 | 0.6385 |
| Median | | 10.0 | 8.0 | 9.0 | | | |
| Range | | 4, 23 | 4, 29 | 3, 67 | | | |
| TOTAL | | | | | | | |
| BILIRUBIN | | | | | | | |
| Change from | | | | | | | |
| Baseline | | | | | | | |
| N | 3.0 DW4 | 16 | 26 | 31 | | | |
| Mean (SD) | | 0.3 (3.11) | −0.8 (4.31) | −4.0 (17.41) | 0.3556 | 0.3370 | 0.3770 |
| Median | | 1.0 | 0.0 | −1.0 | | | |
| Range | | −5, 6 | −17, 7 | −96, 4 | | | |
| N | 4.0 DW8 | 16 | 25 | 29 | | | |
| Mean (SD) | | 2.6 (17.05) | −0.4 (4.78) | −3.8 (13.91) | 0.4109 | 0.1812 | 0.2504 |
| Median | | 0.0 | −1.0 | −1.0 | | | |
| Range | | −24, 61 | −8, 14 | −74, 5 | | | |
| N | 5.0 DW12 | 16 | 24 | 29 | | | |
| Mean (SD) | | 0.8 (7.59) | −1.8 (5.75) | −2.9 (12.93) | 0.2359 | 0.3128 | 0.7088 |
| Median | | 0.0 | −1.0 | 0.0 | | | |
| Range | | −11, 25 | −20, 11 | −65, 16 | | | |
| N | 6.0 DW16 | 15 | 26 | 28 | | | |
| Mean (SD) | | 0.6 (6.90) | −1.3 (9.00) | −3.6 (14.89) | 0.4828 | 0.3040 | 0.4928 |
| Median | | 0.0 | −1.0 | −0.5 | | | |
| Range | | −17, 17 | −32, 17 | −77, 6 | | | |
| N | 7.0 DW20 | 16 | 25 | 29 | | | |
| Mean (SD) | | −0.6 (4.73) | −0.8 (5.69) | −2.7 (14.42) | 0.9375 | 0.5762 | 0.5257 |
| Median | | 0.5 | 0.0 | 0.0 | | | |
| Range | | −16, 5 | −14, 10 | −74, 12 | | | |
| N | 8.0 DW 24 | 16 | 22 | 27 | | | |
| Mean (SD) | | −0.1 (5.12) | −0.5 (7.11) | −2.4 (12.73) | 0.8184 | 0.4805 | 0.5357 |
| Median | | 0.0 | 0.0 | 0.0 | | | |
| Range | | −15, 7 | −22, 9 | −63, 9 | | | |
| UREA NITROGEN | | | | | | | |
| N | 2.0 Baseline | 16 | 34 | 32 | | | |
| Mean (SD) | | 5.8 (1.64) | 5.7 (2.38) | 6.2 (2.16) | 0.9176 | 0.5347 | 0.4226 |
| Median | | 5.9 | 4.9 | 6.1 | | | |
| Range | | 3, 8 | 3, 12 | 2, 13 | | | |
| N | 3.0 DW4 | 16 | 31 | 32 | | | |
| Mean (SD) | | 6.1 (1.63) | 5.3 (2.09) | 6.4 (2.19) | 0.1835 | 0.5914 | 0.0378 |
| Median | | 6.3 | 4.9 | 6.3 | | | |
| Range | | 3, 10 | 3, 11 | 4, 13 | | | |
| N | 4.0 DW8 | 16 | 30 | 31 | | | |
| Mean (SD) | | 5.9 (1.43) | 5.3 (1.58) | 6.4 (2.00) | 0.2159 | 0.4397 | 0.0291 |
| Median | | 5.7 | 5.2 | 6.4 | | | |
| Range | | 4, 8 | 2, 10 | 3, 12 | | | |
| N | 5.0 DW12 | 16 | 28 | 30 | | | |
| Mean (SD) | | 5.9 (1.84) | 4.8 (1.69) | 6.3 (2.08) | 0.0552 | 0.5380 | 0.0049 |
| Median | | 5.9 | 4.4 | 5.9 | | | |
| Range | | 3, 9 | 2, 8 | 3, 13 | | | |
| N | 6.0 DW16 | 15 | 29 | 30 | | | |
| Mean (SD) | | 5.9 (1.24) | 5.0 (1.73) | 6.0 (1.86) | 0.0627 | 0.8182 | 0.0240 |
| Median | | 6.2 | 4.4 | 6.1 | | | |
| Range | | 4, 8 | 3, 9 | 3, 12 | | | |
| N | 7.0 DW20 | 16 | 28 | 29 | | | |
| Mean (SD) | | 5.9 (1.48) | 5.0 (1.80) | 6.3 (1.91) | 0.1319 | 0.4434 | 0.0145 |
| Median | | 6.1 | 5.2 | 6.0 | | | |
| Range | | 3, 9 | 2, 9 | 4, 13 | | | |
| N | 8.0 DW 24 | 16 | 27 | 29 | | | |
| Mean (SD) | | 5.9 (2.28) | 5.0 (1.88) | 6.1 (1.98) | 0.1694 | 0.8151 | 0.0471 |
| Median | | 5.8 | 4.9 | 5.9 | | | |
| Range | | 3, 11 | 1, 9 | 2, 10 | | | |
| UREA NITROGEN | | | | | | | |
| Change from | | | | | | | |
| Baseline | | | | | | | |
| N | 3.0 DW4 | 16 | 30 | 32 | | | |
| Mean (SD) | | 0.3 (1.33) | −0.4 (2.16) | 0.3 (1.22) | 0.2366 | 0.8974 | 0.1392 |
| Median | | −0.1 | −0.4 | 0.2 | | | |
| Range | | −2, 3 | −6, 8 | −2, 3 | | | |
| N | 4.0 DW8 | 16 | 29 | 31 | | | |
| Mean (SD) | | 0.2 (1.34) | −0.4 (2.02) | 0.2 (1.20) | 0.3700 | 0.8901 | 0.1896 |
| Median | | 0.2 | −0.3 | 0.1 | | | |
| Range | | −2, 3 | −7, 3 | −2, 3 | | | |
| N | 5.0 DW12 | 16 | 27 | 30 | | | |
| Mean (SD) | | 0.2 (1.65) | −0.8 (1.86) | 0.1 (1.47) | 0.0898 | 0.8891 | 0.0439 |
| Median | | 0.6 | −0.5 | 0.0 | | | |
| Range | | −4, 3 | −8, 2 | −2, 4 | | | |
| N | 6.0 DW16 | 15 | 28 | 30 | | | |
| Mean (SD) | | 0.3 (1.17) | −0.5 (2.13) | −0.2 (1.47) | 0.1815 | 0.2664 | 0.5093 |
| Median | | 0.2 | −0.3 | 0.0 | | | |
| Range | | −2, 2 | −7, 4 | −4, 3 | | | |
| N | 7.0 DW20 | 16 | 27 | 29 | | | |
| Mean (SD) | | 0.1 (1.54) | −0.6 (1.94) | −0.0 (1.60) | 0.2128 | 0.8544 | 0.1896 |
| Median | | 0.4 | −0.3 | −0.1 | | | |
| Range | | −3, 3 | −6, 3 | −2, 4 | | | |
| N | 8.0 DW 24 | 16 | 26 | 29 | | | |
| Mean (SD) | | 0.1 (2.41) | −0.6 (2.02) | −0.2 (1.87) | 0.2765 | 0.5756 | 0.4506 |
| Median | | 0.1 | −0.7 | 0.3 | | | |
| Range | | −4, 6 | −7, 5 | −4, 4 | | | |

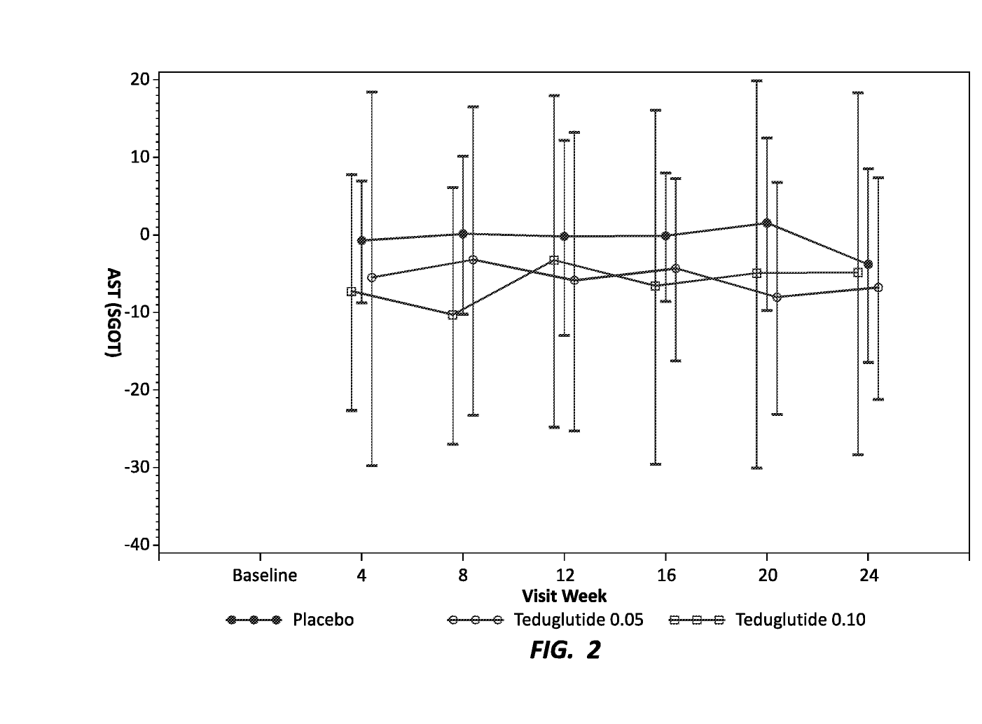

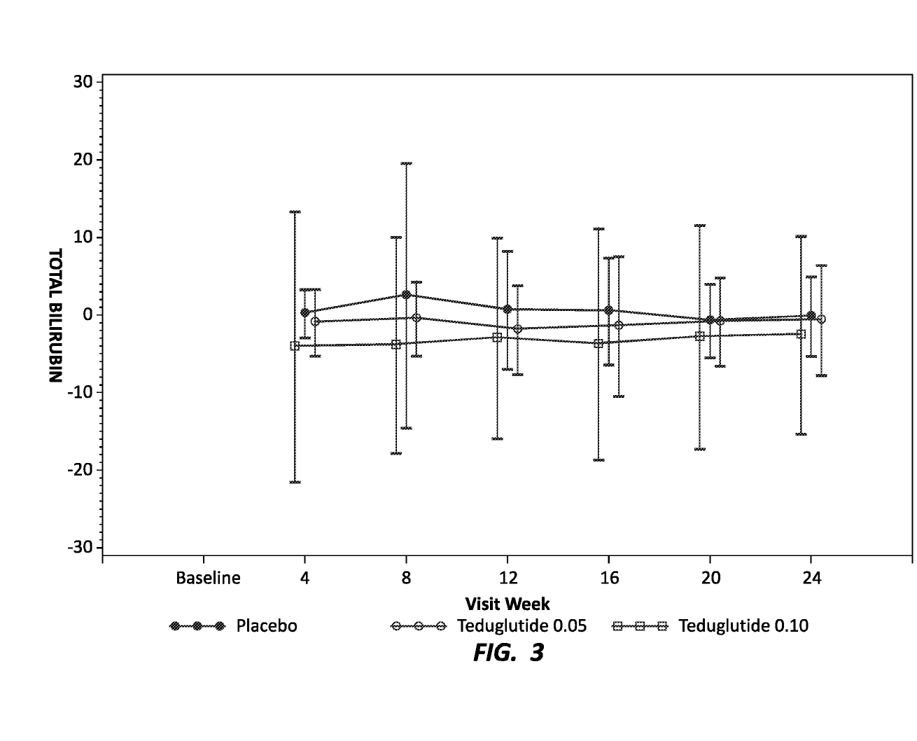

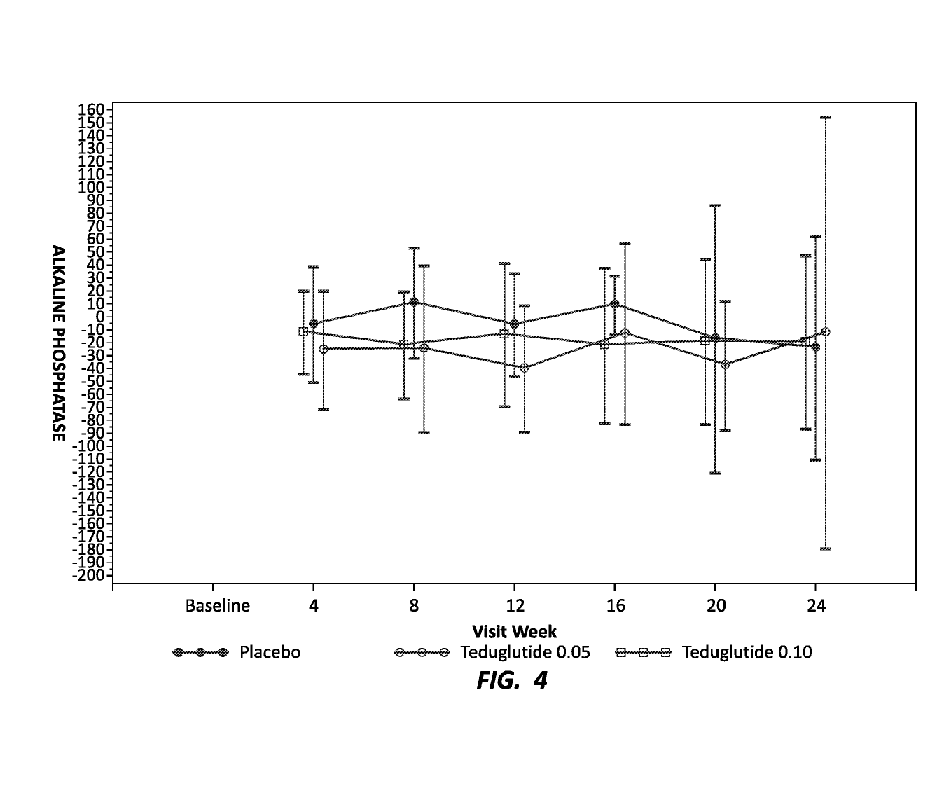

With regards to liver biomarkers, FIG. 1 shows that on average in the groups receiving teduglutide, the level of ALT decreased by about 10 U/L, whereas on average in the group receiving placebo, the level of ALT increased slightly compared to baseline. This reduction in ALT seen in the groups receiving teduglutide was maintained for the entire duration of the study. Similar reductions were seen for AST and total bilirubin (FIGS. 2 and 3 respectively). In the groups receiving teduglutide, the average level of ALP also decreased relative to baseline for the entire period of the study, but in the last two weeks of observation, the average level of the control group also decreased relative to baseline (see FIG. 4). Since several conditions besides liver dysfunction can affect ALP (see Corathers, Pediatrics in Review 27:382, 2006), it is unclear what caused this decrease in the placebo group. However, taken together, this panel of diagnostic biomarkers supports a conclusion that teduglutide helps alleviate liver dysfunction in individuals with short bowel syndrome.

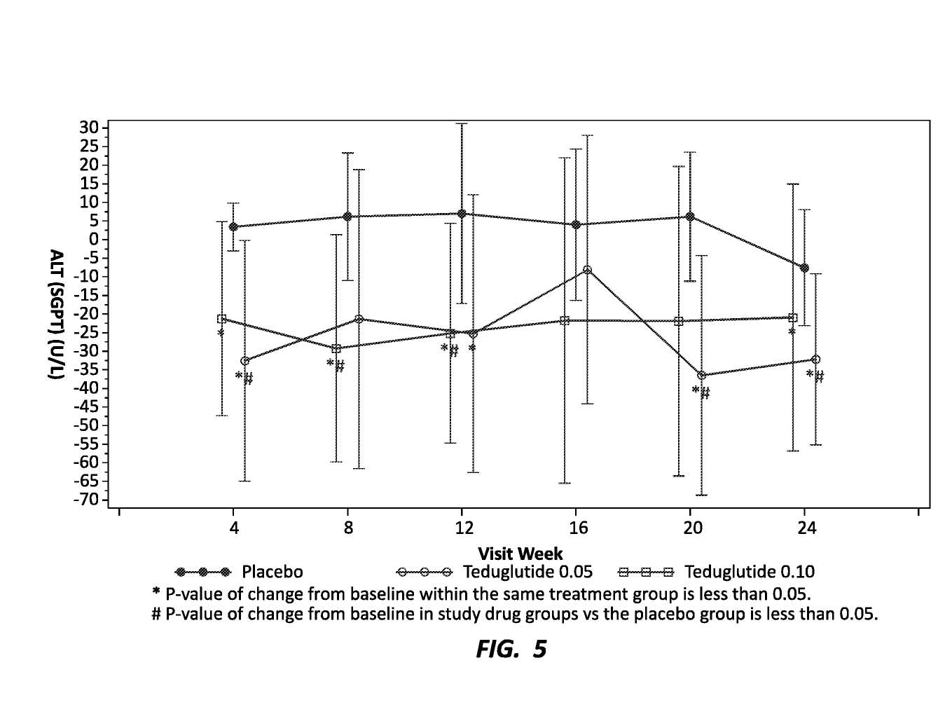

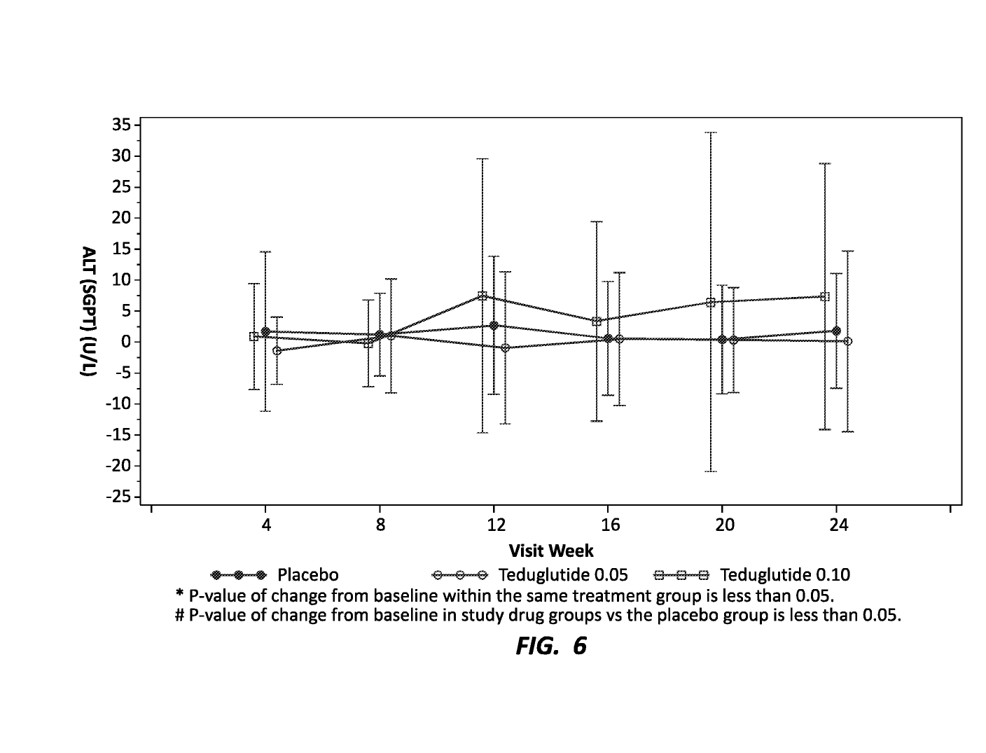

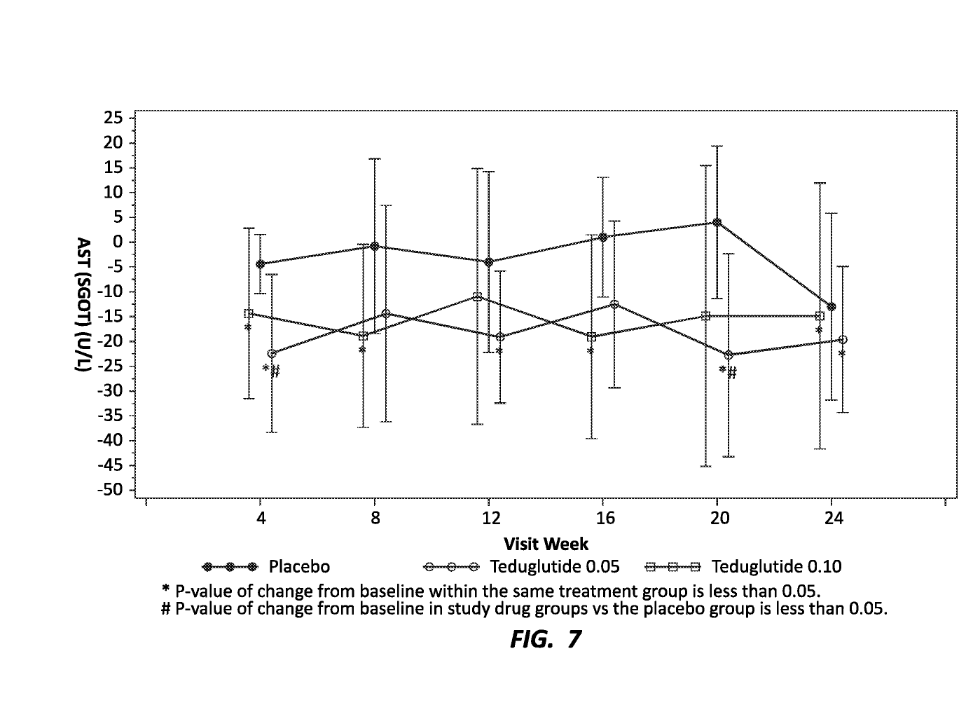

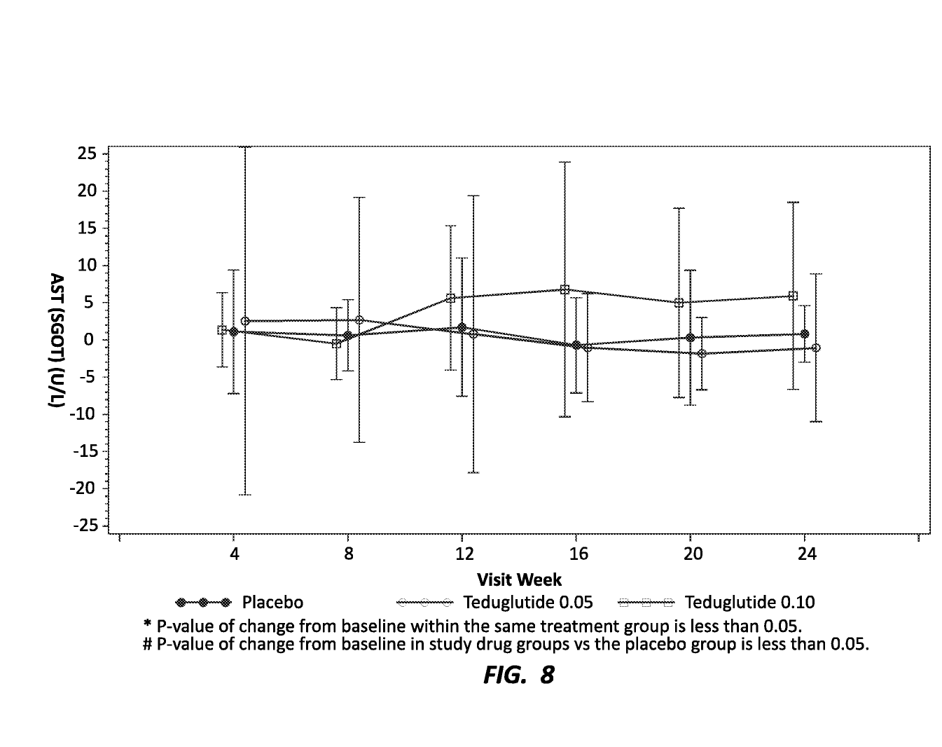

The efficacy of teduglutide in treating liver dysfunction was further tested by sorting the data into two groups: individuals with abnormal baseline values of liver diagnostic biomarkers; and individuals with normal baseline values of liver diagnostic biomarkers. The mean diagnostic biomarker change from baseline for these two groups is shown for ALT (FIGS. 5 and 6) and AST (FIGS. 7 and 8). After treatment with teduglutide, statistically significant improvements for both ALT and AST biomarkers were observed in patients with abnormal baseline values. These changes were significant when compared to baseline levels for the group, and when compared to placebo (see FIGS. 5 and 7).

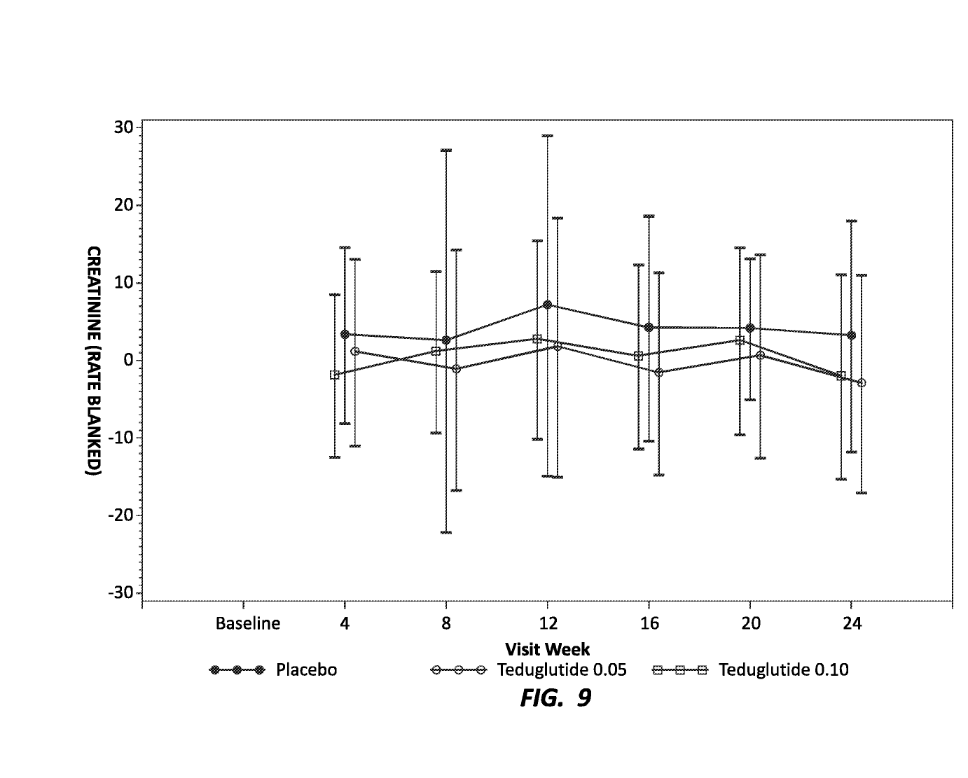

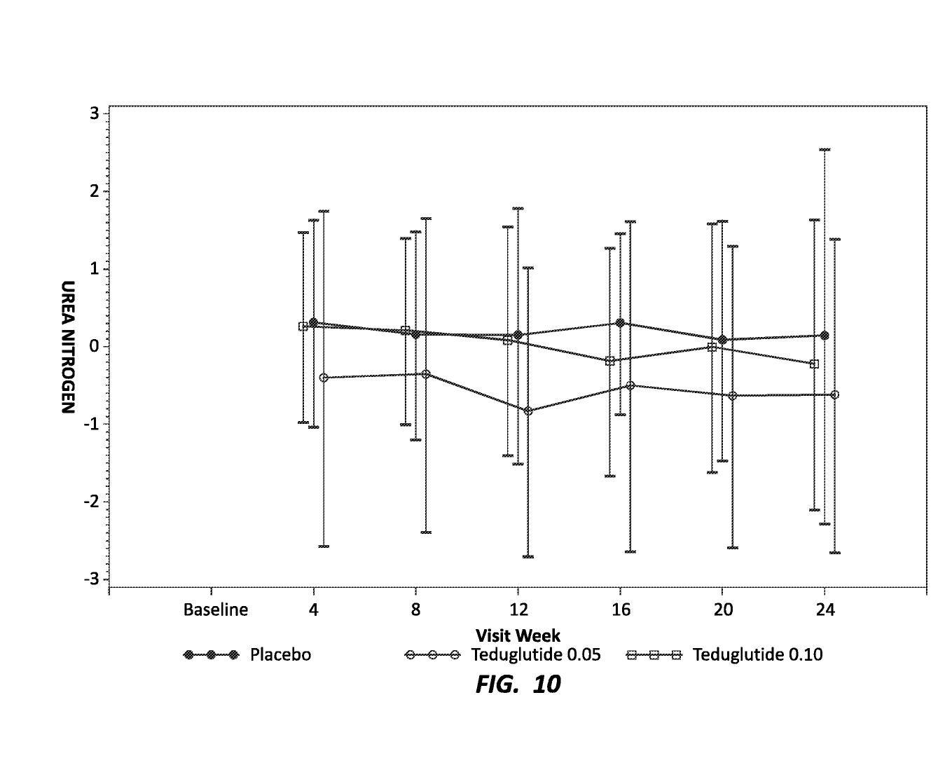

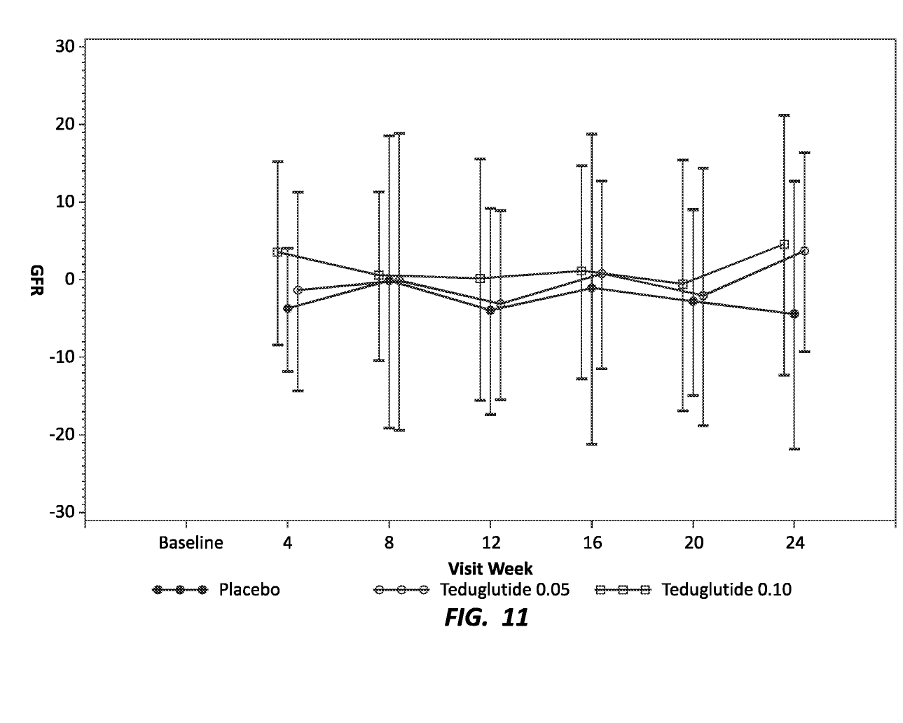

With regards to kidney biomarkers, FIG. 9 shows the average change versus baseline for creatinine. On average, the creatinine level in the group receiving placebo increased relative to baseline, whereas the creatinine levels in the groups receiving teduglutide remained stable or decreased slightly. Similar results are seen in the levels of urea nitrogen, and in the GFR of short bowel patients (see FIGS. 10 and 11).

To further confirm these results, the change in each biomarker for each individual patient was sorted based on whether the biomarker increased more than 5%, decreased more than 5%, or remained within plus or minus 5% from baseline at each time point throughout the course of the study. The raw data for this sorting is reproduced in Table 2 below.

TABLE 2
Selected Lab Tests Change from Baseline

| Lab Tests | Week | Range | Placebo | teduglutide 0.05 | teduglutide 0.1 |
|---|---|---|---|---|---|
| ALKALINE | 4 | −5%<= and | 3(18.8%) | 6(17.1%) | 4(12.5%) |
| PHOS- | | <=5% | | | |
| PHATASE | | | | | |
| | | <−5% | 6(37.5%) | 21(60.0%) | 17(53.1%) |
| | | >5% | 7(43.8%) | 3(8.6%) | 11(34.4%) |
| | 8 | −5%<= and | 3(18.8%) | 2(5.7%) | 2(6.3%) |
| | | <=5% | | | |
| | | <−5% | 5(31.3%) | 21(60.0%) | 24(75.0%) |
| | | >5% | 8(50.0%) | 6(17.1%) | 5(15.6%) |
| | 12 | −5%<= and | 6(37.5%) | 2(5.7%) | 1(3.1%) |
| | | <=5% | | | |
| | | <−5% | 6(37.5%) | 23(65.7%) | 19(59.4%) |
| | | >5% | 4(25.0%) | 2(5.7%) | 10(31.3%) |
| | 16 | −5%<= and | 5(31.3%) | 2(5.7%) | 2(6.3%) |
| | | <=5% | | | |
| | | <−5% | 3(18.8%) | 20(57.1%) | 21(65.6%) |
| | | >5% | 7(43.8%) | 6(17.1%) | 7(21.9%) |
| | 20 | −5%<= and | 3(18.8%) | 1(2.9%) | 1(3.1%) |
| | | <=5% | | | |
| | | <−5% | 4(25.0%) | 22(62.9%) | 19(59.4%) |
| | | >5% | 9(56.3%) | 4(11.4%) | 9(28.1%) |
| | 24 | −5%<= and | 4(25.0%) | 0(0.0%) | 4(12.5%) |
| | | <=5% | | | |
| | | <−5% | 7(43.8%) | 21(60.0%) | 20(62.5%) |
| | | >5% | 5(31.3%) | 4(11.4%) | 5(15.6%) |
| ALT (SGPT) | 4 | −5%<= and | 2(12.5%) | 0(0.0%) | 2(6.3%) |
| | | <=5% | | | |
| | | <−5% | 5(31.3%) | 22(62.9%) | 20(62.5%) |
| | | >5% | 8(50.0%) | 8(22.9%) | 10(31.3%) |
| | 8 | −5%<= and | 1(6.3%) | 4(11.4%) | 2(6.3%) |
| | | <=5% | | | |
| | | <−5% | 6(37.5%) | 16(45.7%) | 20(62.5%) |
| | | >5% | 8(50.0%) | 9(25.7%) | 8(25.0%) |
| | 12 | −5%<= and | 1(6.3%) | 4(11.4%) | 2(6.3%) |
| | | <=5% | | | |
| | | <−5% | 7(43.8%) | 19(54.3%) | 20(62.5%) |
| | | >5% | 7(43.8%) | 4(11.4%) | 7(21.9%) |
| | 16 | −5%<= and | 0(0.0%) | 0(0.0%) | 2(6.3%) |
| | | <=5% | | | |
| | | >−5% | 8(50.0%) | 16(45.7%) | 19(59.4%) |
| | | >5% | 7(43.8%) | 12(34.3%) | 9(28.1%) |
| | 20 | −5%<= and | 3(18.8%) | 1(2.9%) | 2(6.3%) |
| | | <=5% | | | |
| | | <−5% | 6(37.5%) | 17(48.6%) | 19(59.4%) |
| | | >5% | 6(37.5%) | 9(25.7%) | 8(25.0%) |
| | 24 | −5%<= and | 1(6.3%) | 4(11.4%) | 0(0.0%) |
| | | <=5% | | | |
| | | <−5% | 8(50.0%) | 18(51.4%) | 18(56.3%) |
| | | >5% | 6(37.5%) | 4(11.4%) | 11(34.4%) |
| AST (SGOT) | 4 | −5%<= and | 3(18.8%) | 2(5.7%) | 4(12.5%) |
| | | <=5% | | | |
| | | <−5% | 7(43.8%) | 21(60.0%) | 17(53.1%) |
| | | >5% | 5(31.3%) | 5(14.3%) | 10(31.3%) |
| | 8 | −5%<= and | 1(6.3%) | 4(11.4%) | 4(12.5%) |
| | | <=5% | | | |
| | | <−5% | 6(37.5%) | 16(45.7%) | 21(65.6%) |
| | | >5% | 8(50.0%) | 9(25.7%) | 5(15.6%) |
| | 12 | −5%<= and | 0(0.0%) | 3(8.6%) | 3(9.4%) |
| | | <=5% | | | |
| | | <−5% | 8(50.0%) | 19(54.3%) | 15(46.9%) |
| | | >5% | 7(43.8%) | 5(14.3%) | 10(31.3%) |
| | 16 | −5%<= and | 1(6.3%) | 6(17.1%) | 3(9.4%) |
| | | <=5% | | | |
| | | <−5% | 8(50.0%) | 14(40.0%) | 18(56.3%) |
| | | >5% | 6(37.5%) | 8(22.9%) | 8(25.0%) |
| | 20 | −5%<= and | 0(0.0%) | 4(11.4%) | 3(9.4%) |
| | | <=5% | | | |
| | | <−5% | 9(56.3%) | 17(48.6%) | 16(50.0%) |
| | | >5% | 6(37.5%) | 6(17.1%) | 9(28.1%) |
| | 24 | −5%<= and | 4(25.0%) | 3(8.6%) | 3(9.4%) |
| | | <=5% | | | |
| | | <−5% | 6(37.5%) | 17(48.6%) | 15(46.9%) |
| | | >5% | 5(31.3%) | 6(17.1%) | 11(34.4%) |
| CREATININE | 4 | −5%<= and | 5(31.3%) | 9(25.7%) | 13(40.6%) |
| (RATE | | <=5% | | | |
| BLANKED) | | | | | |
| | | <−5% | 3(18.8%) | 7(20.0%) | 12(37.5%) |
| | | >5% | 8(50.0%) | 14(40.0%) | 7(21.9%) |
| | 8 | −5%<= and | 7(43.8%) | 8(22.9%) | 10(31.3%) |
| | | <=5% | | | |
| | | <−5% | 4(25.0%) | 11(31.4%) | 8(25.0%) |
| | | >5% | 5(31.3%) | 10(28.6%) | 13(40.6%) |
| | 12 | −5%<= and | 4(25.0%) | 4(11.4%) | 4(12.5%) |
| | | <=5% | | | |
| | | <−5% | 4(25.0%) | 7(20.0%) | 9(28.1%) |
| | | >5% | 8(50.0%) | 16(45.7%) | 17(53.1%) |
| | 16 | −5%<= and | 2(12.5%) | 12(34.3%) | 8(25.0%) |
| | | <=5% | | | |
| | | <−5% | 5(31.3%) | 8(22.9%) | 10(31.3%) |
| | | >5% | 8(50.0%) | 8(22.9%) | 12(37.5%) |
| | 20 | −5%<= and | 4(25.0%) | 8(22.9%) | 9(28.1%) |
| | | <=5% | | | |
| | | <−5% | 3(18.8%) | 9(25.7%) | 7(21.9%) |
| | | >5% | 9(56.3%) | 10(28.6%) | 13(40.6%) |
| | 24 | −5%<= and | 3(18.8%) | 9(25.7%) | 9(28.1%) |
| | | <=5% | | | |
| | | <−5% | 5(31.3%) | 8(22.9%) | 11(34.4%) |
| | | >5% | 8(50.0%) | 9(25.7%) | 9(28.1%) |
| GFR | 4 | −5%<= and | 6(37.5%) | 9(25.7%) | 11(34.4%) |
| | | <=5% | | | |
| | | <−5% | 7(43.8%) | 13(37.1%) | 7(21.9%) |
| | | >5% | 3(18.8%) | 8(22.9%) | 14(43.8%) |
| | 8 | −5%<= and | 6(37.5%) | 7(20.0%) | 13(40.6%) |
| | | <=5% | | | |
| | | <−5% | 5(31.3%) | 9(25.7%) | 9(28.1%) |
| | | >5% | 5(31.3%) | 12(34.3%) | 9(28.1%) |
| | 12 | −5%<= and | 4(25.0%) | 5(14.3%) | 10(31.3%) |
| | | <=5% | | | |
| | | <−5% | 8(50.0%) | 15(42.9%) | 11(34.4%) |
| | | >5% | 4(25.0%) | 6(17.1%) | 9(28.1%) |
| | 16 | −5%<= and | 4(25.0%) | 10(28.6%) | 6(18.8%) |
| | | <=5% | | | |
| | | <−5% | 8(50.0%) | 7(20.0%) | 12(37.5%) |
| | | >5% | 2(12.5%) | 11(31.4%) | 12(37.5%) |
| | 20 | −5%<= and | 3(18.8%) | 7(20.0%) | 10(31.3%) |
| | | <=5% | | | |
| | | <−5% | 10(62.5%) | 10(28.6%) | 11(34.4%) |
| | | >5% | 3(18.8%) | 10(28.6%) | 8(25.0%) |
| | 24 | −5%<= and | 6(37.5%) | 8(22.9%) | 9(28.1%) |
| | | <=5% | | | |
| | | <−5% | 6(37.5%) | 8(22.9%) | 6(18.8%) |
| | | >5% | 4(25.0%) | 10(28.6%) | 14(43.8%) |
| TOTAL | 4 | −5%<= and | 1(6.3%) | 6(17.1%) | 3(9.4%) |
| BILIRUBIN | | <=5% | | | |
| | | <−5% | 6(37.5%) | 12(34.3%) | 16(50.0%) |
| | | >5% | 9(56.3%) | 8(22.9%) | 12(37.5%) |
| | 8 | −5%<= and | 4(25.0%) | 4(11.4%) | 6(18.8%) |
| | | <=5% | | | |
| | | <−5% | 7(43.8%) | 13(37.1%) | 16(50.0%) |
| | | >5% | 5(31.3%) | 8(22.9%) | 7(21.9%) |
| | 12 | −5%<= and | 4(25.0%) | 4(11.4%) | 6(18.8%) |
| | | <=5% | | | |
| | | <−5% | 6(37.5%) | 14(40.0%) | 14(43.8%) |
| | | >5% | 6(37.5%) | 6(17.1%) | 9(28.1%) |
| | 16 | −5%<= and | 4(25.0%) | 3(8.6%) | 8(25.0%) |
| | | <=5% | | | |
| | | <−5% | 5(31.3%) | 14(40.0%) | 14(43.8%) |
| | | >5% | 6(37.5%) | 9(25.7%) | 6(18.8%) |
| | 20 | −5%<= and | 3(18.8%) | 6(17.1%) | 6(18.8%) |
| | | <=5% | | | |
| | | <−5% | 5(31.3%) | 12(34.3%) | 14(43.8%) |
| | | >5% | 8(50.0%) | 7(20.0%) | 9(28.1%) |
| | 24 | −5%<= and | 5(31.3%) | 4(11.4%) | 6(18.8%) |
| | | <=5% | | | |
| | | <−5% | 4(25.0%) | 8(22.9%) | 11(34.4%) |
| | | >5% | 7(43.8%) | 10(28.6%) | 10(31.3%) |
| UREA | 4 | −5%<= and | 3(18.8%) | 7(20.0%) | 7(21.9%) |
| NITROGEN | | <=5% | | | |
| | | <−5% | 6(37.5%) | 16(45.7%) | 10(31.3%) |
| | | >5% | 7(43.8%) | 7(20.0%) | 15(46.9%) |
| | 8 | −5%<= and | 2(12.5%) | 5(14.3%) | 7(21.9%) |
| | | <=5% | | | |
| | | <−5% | 6(37.5%) | 14(40.0%) | 9(28.1%) |
| | | >5% | 8(50.0%) | 10(28.6%) | 15(46.9%) |
| | 12 | −5%<= and | 1(6.3%) | 4(11.4%) | 3(9.4%) |
| | | <=5% | | | |
| | | <−5% | 6(37.5%) | 17(48.6%) | 13(40.6%) |
| | | >5% | 9(56.3%) | 6(17.1%) | 14(43.8%) |
| | 16 | −5%<= and | 3(18.8%) | 6(17.1%) | 12(37.5%) |
| | | <=5% | | | |
| | | <−5% | 5(31.3%) | 12(34.3%) | 9(28.1%) |
| | | >5% | 7(43.8%) | 10(28.6%) | 9(28.1%) |
| | 20 | −5%<= and | 2(12.5%) | 7(20.0%) | 6(18.8%) |
| | | <=5% | | | |
| | | <−5% | 6(37.5%) | 13(37.1%) | 12(37.5%) |
| | | >5% | 8(50.0%) | 7(20.0%) | 11(34.4%) |
| | 24 | −5%<= and | 2(12.5%) | 2(5.7%) | 2(6.3%) |
| | | <=5% | | | |
| | | <−5% | 7(43.8%) | 7(48.6%) | 14(43.8%) |
| | | >5% | 7(43.8%) | 7(20.0%) | 13(40.6%) |

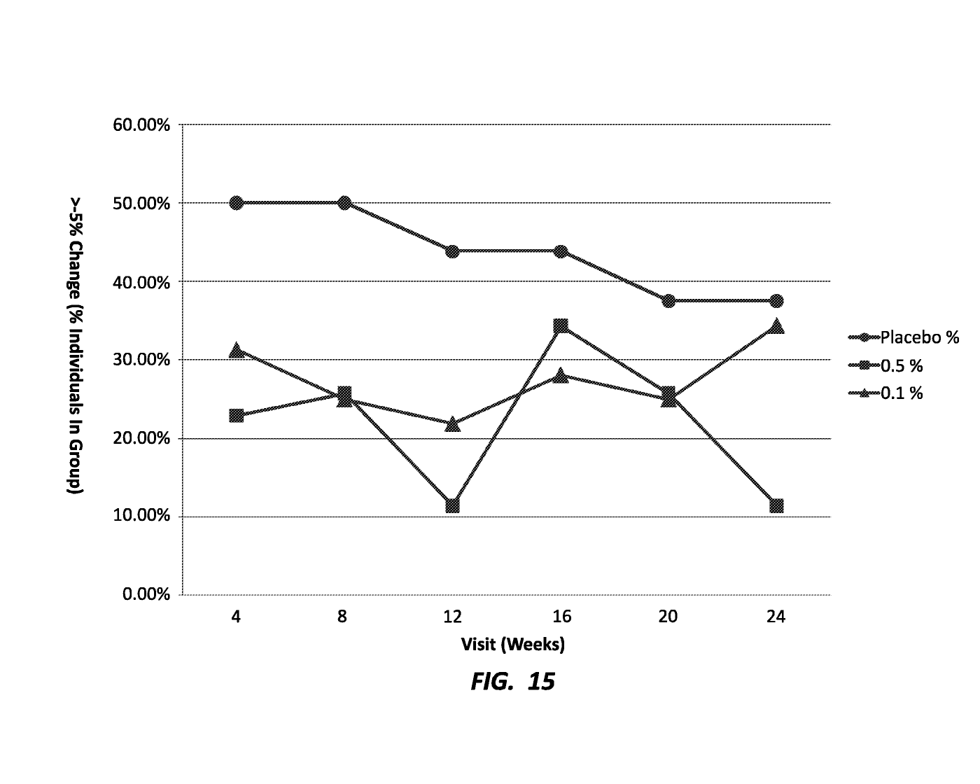

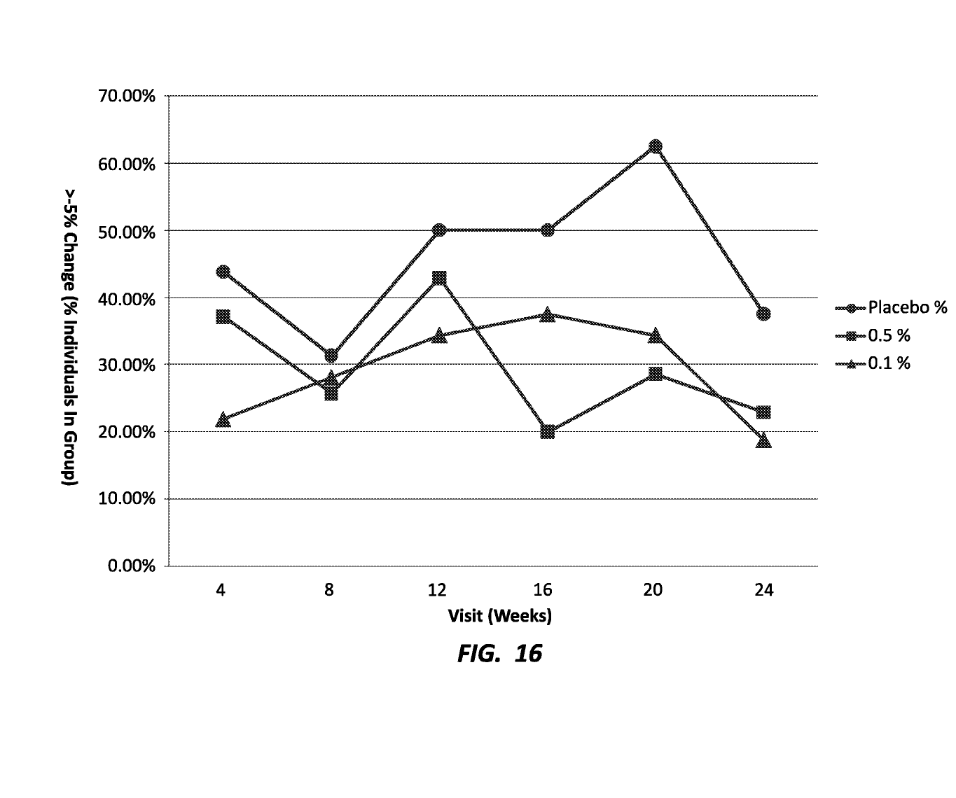

For illustration, FIGS. 15 and 16 show a compilation of the data from Table 2 for ALP and ALT (liver disease biomarkers) over the course of the study. FIG. 15 shows that within 4 weeks of the beginning of treatment, roughly 60% of the individuals in the groups receiving teduglutide experienced a decrease in ALP greater than 5% from baseline, whereas the percentage of individuals from the placebo group experiencing a similar decrease is much smaller. A similar dataset for ALT confirms that, in general, groups receiving teduglutide had a greater fraction of individuals experiencing a decrease of more than 5% in liver biomarkers associated with liver disease than groups receiving placebo, beginning at 4 weeks after the commencement of treatment.

By comparing the percent of individuals experiencing an increase in biomarkers associated with disease between groups, it is possible to determine whether GLP-2 can be effective in prophylaxis as well as treatment of liver and kidney disease associated with SBS, PN and intestinal failure.

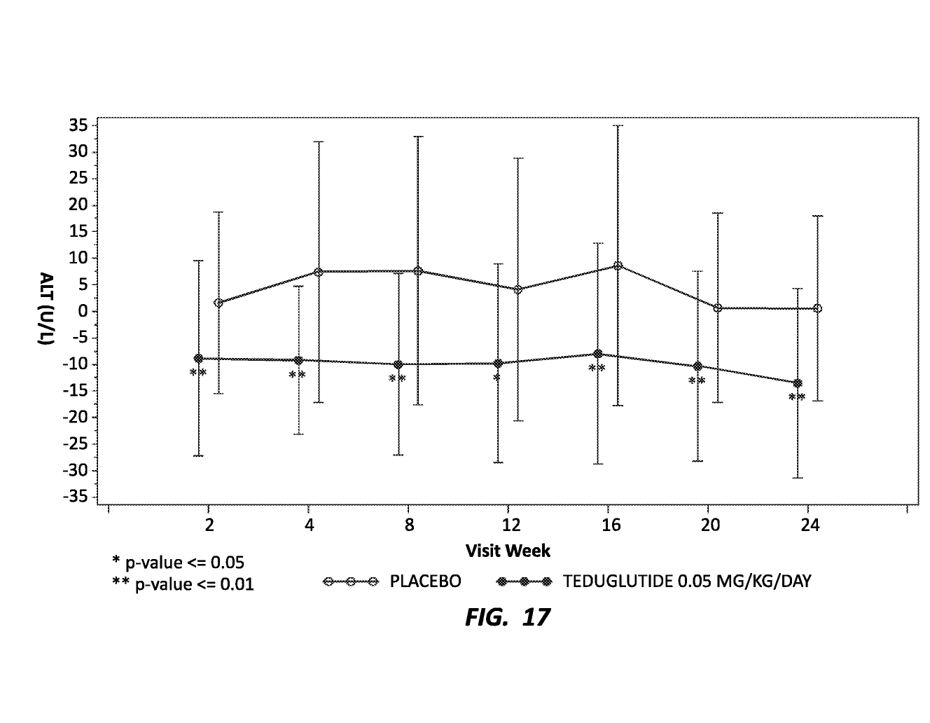

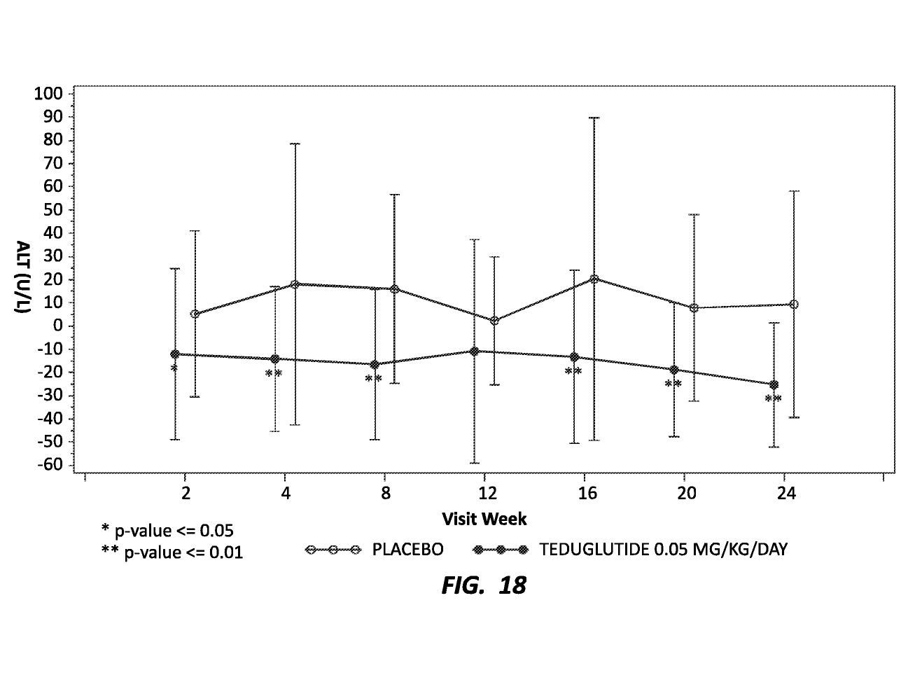

FIG. 17 compares the percent of individuals in the group receiving placebo who experienced an increase of greater than 5% in ALP to the percent of individuals experiencing the same increase in the groups receiving teduglutide. FIG. 18 shows the same type of data for ALT. These graphs show that, in general, the percent of individuals receiving placebo who experienced an increase in biomarkers associated with liver disease is greater than the percent of individuals receiving teduglutide who experienced the same increase. These results support the conclusion that teduglutide is an effective prophylactic against increases in biomarkers associated with liver dysfunction when administered to individuals with SBS.

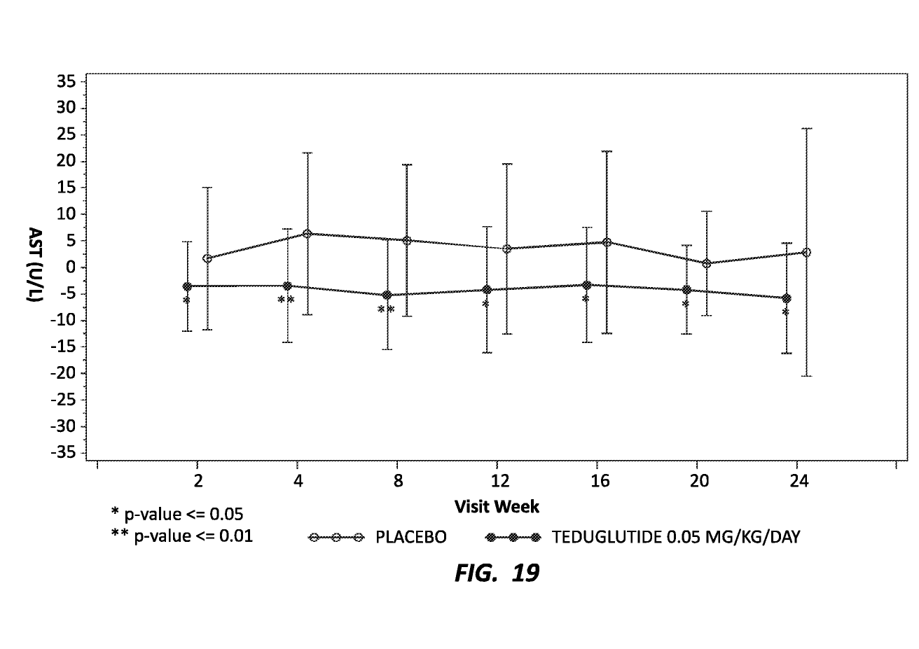

A similar prophylactic effect is seen for kidneys in FIG. 19. FIG. 19 compares the percent of the placebo group experiencing a decrease in GFR (which is linked with decreased kidney function) to the percent of individuals experiencing the same decrease in the groups receiving teduglutide. In both groups receiving teduglutide, the percent of the group experiencing a decrease in GFR greater than 5% is always less than the percent of the group receiving placebo that experience the same decrease.

### Example 2

To further confirm the results of Example 1, a further study was conducted involving more individuals and additional diagnostic biomarkers. Human patients with short bowel syndrome were divided into two groups: a control group (43 total individuals) to receive placebo, and an experimental group (42 total individuals) to receive teduglutide at 0.05 mg/kg/day throughout the study. Patients were treated by subcutaneous injection of placebo or the appropriate dose of teduglutide. Known biomarkers of liver function were monitored every four weeks, for 24 weeks. The liver biomarkers tested included albumin, gamma glutamyl transferase, total bilirubin, ALT and AST and ALP. Before administration of placebo or teduglutide, diagnostic biomarker levels were tested to establish a baseline for each individual.

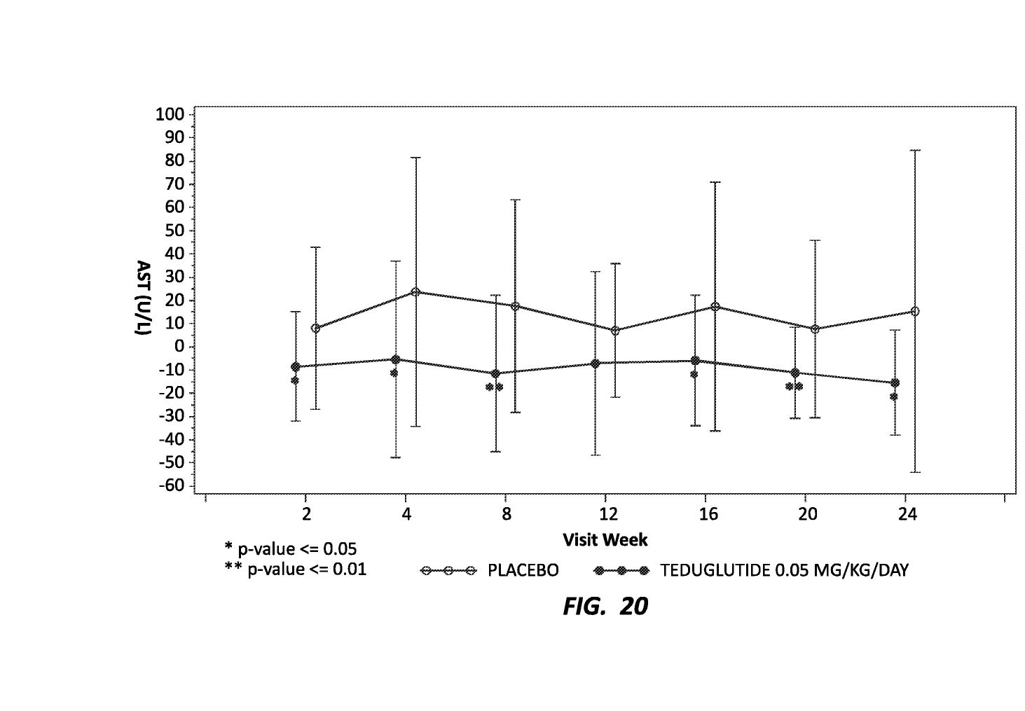

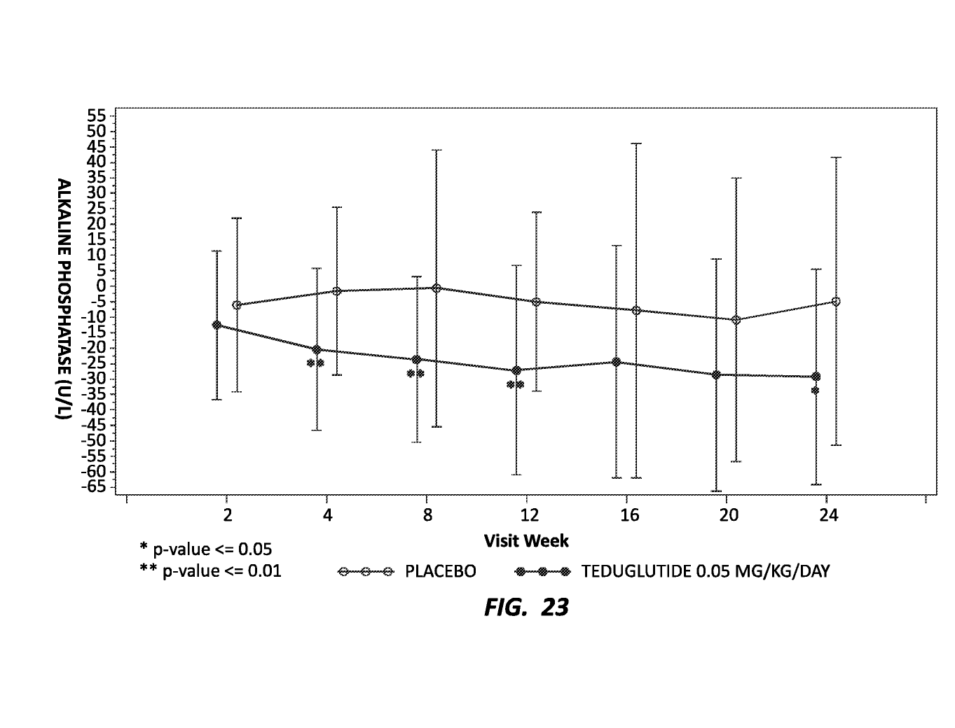

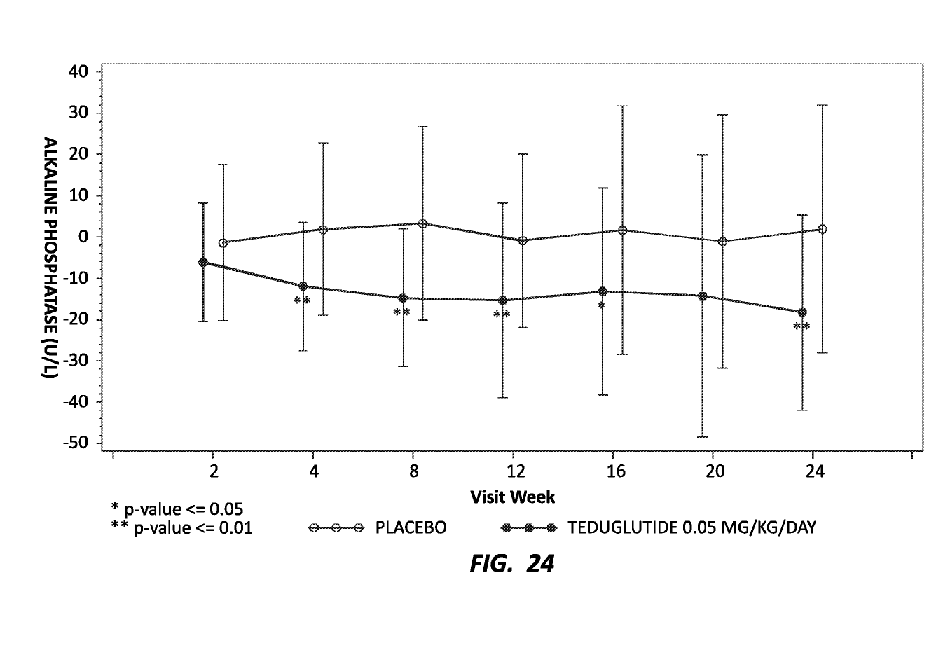

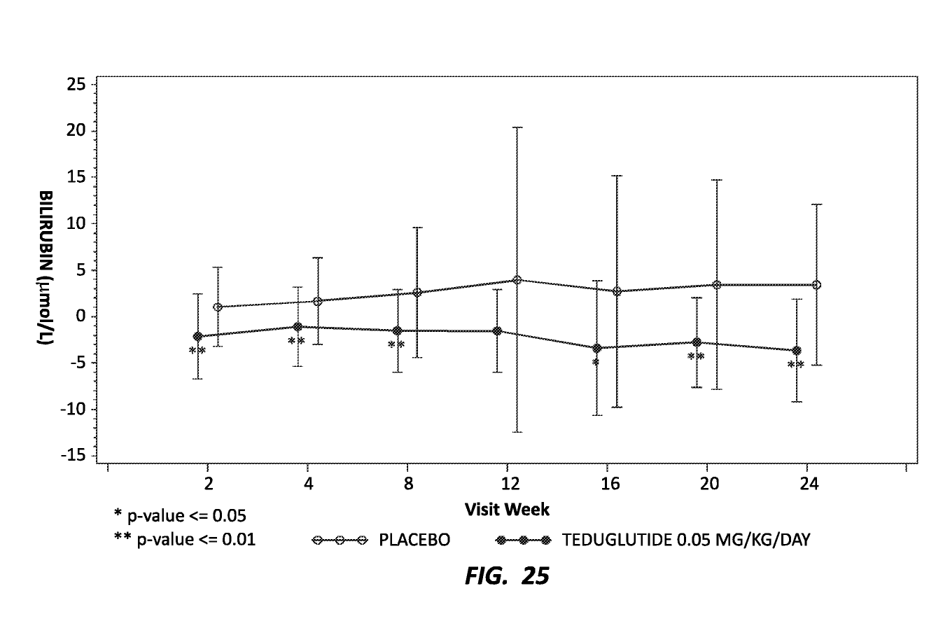

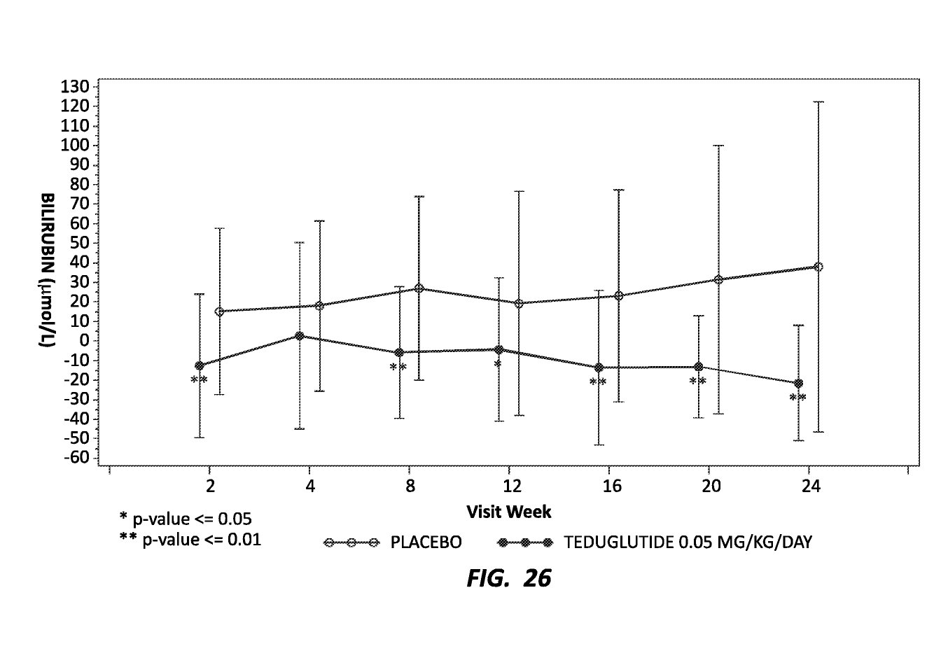

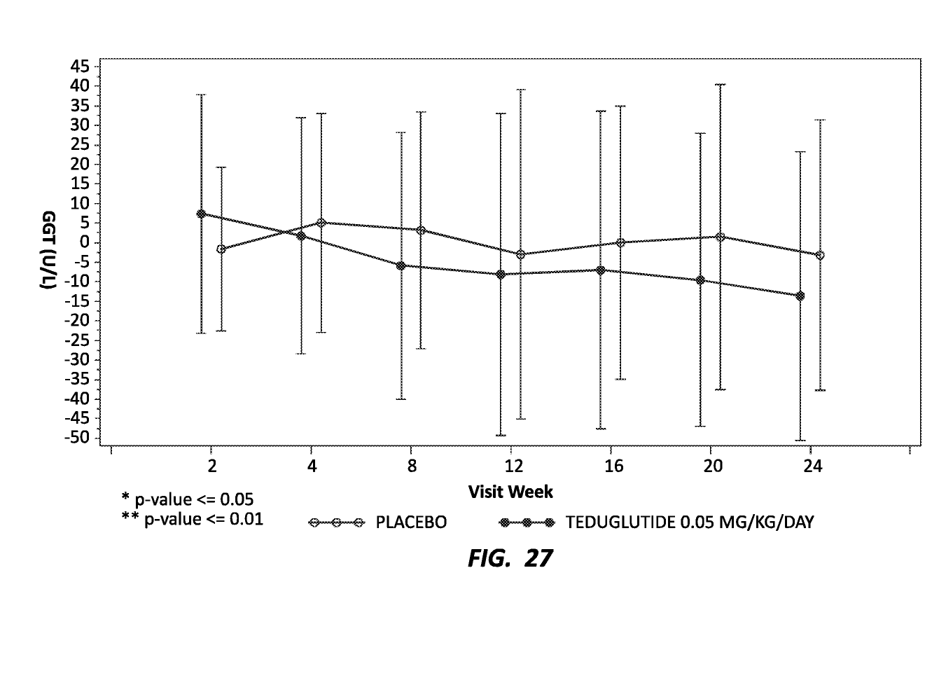

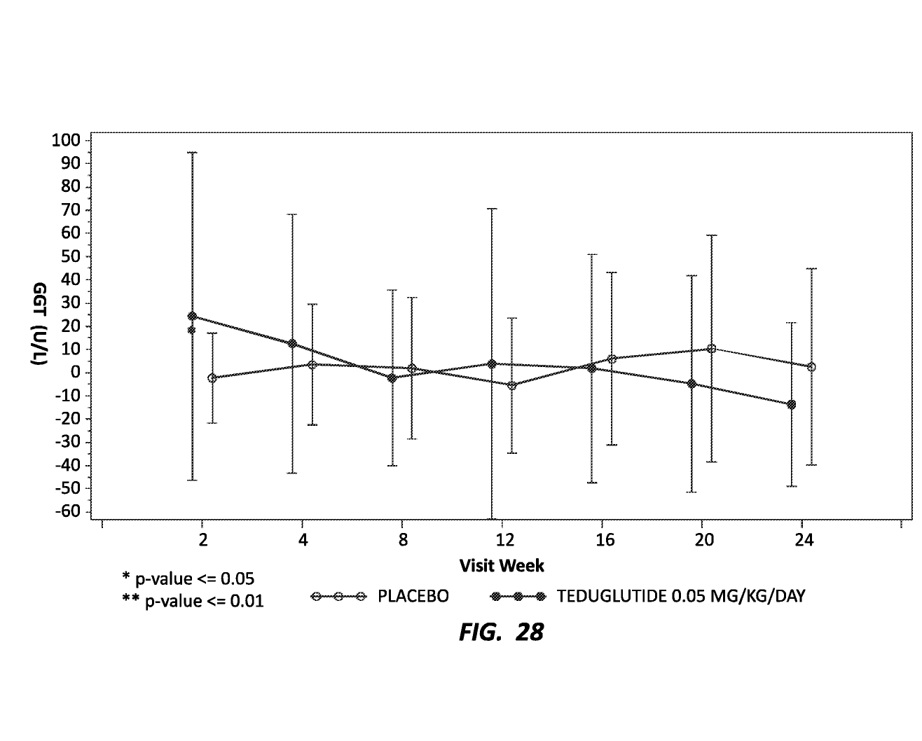

To determine the effect of GLP-2 on the levels of the diagnostic biomarkers, the average level of each biomarker was measured for the placebo and teduglutide treated groups for each visit. Furthermore, the average change from baseline was calculated using the established baseline values. The average level and average change from baseline for each biomarker of each group is given in Table 3. Examples of the changes observed are provided in FIGS. 17 through 28. For FIGS. 17-28, p-values were calculated using the t-test.

TABLE 3
Selected Lab Test Results

| | | | | | | Teduglutide 0.05 | | | |
|---|---|---|---|---|---|---|---|---|---|
| | | Placebo (N = 43) | | | | mg/kg/d (N = 42) | | | |
| | | | | Change | | | | Change | |
| | | Observed | | from | | Observed | | from | |
| Parameter/Visit | Statistic | Value | | Baseline | | Value | | Baseline | |
| Albumin (g/L) | | | | | | | | | |
| Baseline | n | 43 | | | | 42 | | | |
| | Mean (SD) | 41.8 | (4.1) | | | 42.4 | (3.2) | | |
| | Median | 42 | | | | 42.5 | | | |
| | Min, Max | 31, 51 | | | | 35, 48 | | | |
| Week 2 | n | 43 | | 43 | | 40 | | 40 | |
| | Mean (SD) | 41.0 | (4.2) | −0.8 | (3.0) | 41.5 | (3.0) | −0.9 | (2.0) |
| | Median | 41 | | −1 | | 42 | | −1 | |
| | Min, Max | 32, 49 | | −12, 6 | | 35, 47 | | −5, 3 | |
| Week 4 | n | 43 | | 43 | | 40 | | 40 | |
| | Mean (SD) | 41.1 | (3.9) | −0.7 | (2.1) | 41.7 | (3.0) | −0.7 | (2.6) |
| | Median | 42 | | −1 | | 42 | | −1 | |
| | Min, Max | 30, 47 | | −6, 3 | | 34, 47 | | −7, 5 | |
| Week 8 | n | 41 | | 41 | | 40 | | 40 | |
| | Mean (SD) | 41.4 | (4.7) | −0.6 | (2.4) | 41.7 | (3.2) | −0.7 | (2.1) |
| | Median | 42 | | −1 | | 42 | | −1 | |
| | Min, Max | 31, 54 | | −5, 7 | | 36, 48 | | −5, 4 | |
| Week 12 | n | 33 | | 33 | | 34 | | 34 | |
| | Mean (SD) | 40.6 | (4.1) | −1.2 | (1.9) | 41.2 | (3.2) | −0.9 | (2.4) |
| | Median | 41 | | −1 | | 41 | | −1 | |
| | Min, Max | 33, 50 | | −6, 4 | | 35, 47 | | −7, 5 | |
| Week 16 | n | 40 | | 40 | | 38 | | 38 | |
| | Mean (SD) | 40.8 | (4.3) | −1.3 | (2.1) | 41.7 | (3.4) | −0.7 | (2.1) |
| | Median | 41.5 | | −1 | | 42 | | −0.5 | |
| | Min, Max | 31, 48 | | −7, 3 | | 32, 49 | | −5, 3 | |
| Week 20 | n | 40 | | 40 | | 38 | | 38 | |
| | Mean (SD) | 40.7 | (4.2) | −1.3 | (2.5) | 41.8 | (4.0) | −0.6 | (2.4) |
| | Median | 41 | | −1.5 | | 43 | | −1 | |
| | Min, Max | 32, 50 | | −6, 5 | | 33, 49 | | −8, 4 | |
| Week 24 | n | 39 | | 39 | | 38 | | 38 | |
| | Mean (SD) | 40.1 | (4.4) | −1.7 | (2.4) | 41.9 | (3.2) | −0.4 | (2.3) |
| | Median | 40 | | −2 | | 42 | | 0 | |
| | Min, Max | 31, 49 | | −6, 4 | | 33, 48 | | −8, 3 | |
| Endpoint | n | 43 | | 43 | | 42 | | 42 | |
| | Mean (SD) | 40.3 | (4.4) | −1.5 | (2.5) | 41.9 | (3.2) | −0.5 | (2.3) |
| | Median | 41 | | −2 | | 42 | | 0 | |
| | Min, Max | 31, 49 | | −6, 4 | | 33, 48 | | −8, 3 | |
| Alkaline | | | | | | | | | |
| Phosphatase (U/L) | | | | | | | | | |
| Baseline | n | 43 | | | | 42 | | | |
| | Mean (SD) | 151.8 | (85.3) | | | 134.7 | (66.3) | | |
| | Median | 124 | | | | 112 | | | |
| | Min, Max | 40, 506 | | | | 50, 351 | | | |
| Week 2 | n | 43 | | 43 | | 40 | | 40 | |
| | Mean (SD) | 145.7 | (84.0) | −6.1 | (28.1) | 123.7 | (54.7) | −12.5 | (23.9) |
| | Median | 121 | | −1 | | 104 | | −8 | |
| | Min, Max | 62, 547 | | −94, 41 | | 53, 298 | | −76, 27 | |
| Week 4 | n | 42 | | 42 | | 40 | | 40 | |
| | Mean (SD) | 148.8 | (82.2) | −1.6 | (27.1) | 115.8 | (52.7) | −20.5 | (26.2) |
| | Median | 128 | | −1.5 | | 101 | | −15.5 | |
| | Min, Max | 60, 474 | | −102, 59 | | 54, 307 | | −89, 34 | |
| Week 8 | n | 40 | | 40 | | 40 | | 40 | |
| | Mean (SD) | 152.3 | (80.2) | −0.7 | (44.7) | 112.0 | (56.4) | −24.3 | (26.9) |
| | Median | 130 | | 4 | | 94 | | −18.5 | |
| | Min, Max | 59, 365 | | −141, 104 | | 46, 341 | | −105, 19 | |
| Week 12 | n | 33 | | 33 | | 34 | | 34 | |
| | Mean (SD) | 150.5 | (86.5) | −5.1 | (29.0) | 108.6 | (54.7) | −27.2 | (33.9) |
| | Median | 122 | | −6 | | 98 | | −23 | |
| | Min, Max | 49, 483 | | −64, 67 | | 52, 304 | | −127, 25 | |
| Week 16 | n | 40 | | 40 | | 38 | | 38 | |
| | Mean (SD) | 144.5 | (65.1) | −7.8 | (54.1) | 114.2 | (55.9) | −24.5 | (37.5) |
| | Median | 129.5 | | −3 | | 102 | | −22.5 | |
| | Min, Max | 49, 320 | | −224, 116 | | 46, 311 | | −104, 71 | |
| Week 20 | n | 40 | | 40 | | 38 | | 38 | |
| | Mean (SD) | 141.5 | (67.7) | −10.9 | (45.9) | 109.8 | (52.1) | −28.6 | (37.6) |
| | Median | 125 | | −5.5 | | 94.5 | | −25.5 | |
| | Min, Max | 54, 364 | | −142, 90 | | 54, 303 | | −117, 77 | |
| Week 24 | n | 39 | | 39 | | 38 | | 38 | |
| | Mean (SD) | 143.3 | (71.6) | −4.9 | (46.4) | 106.2 | (59.6) | −29.2 | (34.8) |
| | Median | 124 | | −1 | | 88 | | −24.5 | |
| | Min, Max | 54, 383 | | −137, 94 | | 49, 328 | | −122, 33 | |
| Endpoint | n | 43 | | 43 | | 42 | | 42 | |
| | Mean (SD) | 145.2 | (70.6) | −6.6 | (46.6) | 106.4 | (56.9) | −28.3 | (34.9) |
| | Median | 129 | | −1 | | 90.5 | | −22 | |
| | Min, Max | 54, 383 | | −137, 94 | | 49, 328 | | −122, 33 | |
| ALT (U/L) | | | | | | | | | |
| Baseline | n | 43 | | | | 42 | | | |
| | Mean (SD) | 44.2 | (35.0) | | | 43.0 | (29.1) | | |
| | Median | 36 | | | | 33 | | | |
| | Min, Max | 12, 219 | | | | 11, 143 | | | |
| Week 2 | n | 43 | | 43 | | 40 | | 40 | |
| | Mean (SD) | 45.8 | (43.9) | 1.6 | (17.0) | 35.2 | (24.7) | −8.9 | (18.4) |
| | Median | 33 | | −2 | | 27.5 | | −5.5 | |
| | Min, Max | 14, 289 | | | −25, 70 | 9, 142 | | −58, 48 | |
| Week 4 | n | 43 | | 43 | | 40 | | 40 | |
| | Mean (SD) | 51.6 | (46.6) | 7.4 | (24.6) | 34.9 | (21.2) | −9.2 | (14.0) |
| | Median | 39 | | 2 | | 29 | | −7 | |
| | Min, Max | 14, 280 | | −30, 92 | | 8, 104 | | −44, 29 | |
| Week 8 | n | 41 | | 41 | | 40 | | 40 | |
| | Mean (SD) | 51.2 | (51.8) | 7.6 | (25.2) | 33.9 | (24.1) | −10.2 | (17.3) |
| | Median | 34 | | 3 | | 26 | | −5.5 | |
| | Min, Max | 16, 324 | | 53, 105 | | 9, 138 | | −65, 20 | |
| Week 12 | n | 33 | | 33 | | 34 | | 34 | |
| | Mean (SD) | 50.6 | (59.3) | 4.1 | (24.7) | 33.6 | (19.6) | −9.8 | (18.7) |
| | Median | 37 | | 2 | | 27.5 | | −4 | |
| | Min, Max | 17, 339 | | −40, 120 | | 11, 94 | | −62, 35 | |
| Week 16 | n | 40 | | 40 | | 38 | | 38 | |
| | Mean (SD) | 52.8 | (52.5) | 8.6 | (26.3) | 37.5 | (29.4) | −8.0 | (20.8) |
| | Median | 33.5 | | 2 | | 26.5 | | −3 | |
| | Min, Max | 14, 296 | | −36, 84 | | 13, 140 | | −64, 45 | |
| Week 20 | n | 40 | | 40 | | 38 | | 38 | |
| | Mean (SD) | 44.8 | (39.7) | 0.7 | (17.9) | 34.8 | (26.7) | −10.3 | (17.8) |
| | Median | 35 | | 0.5 | | 28.5 | | −6.5 | |
| | Min, Max | 16, 253 | | −66, 52 | | 5, 149 | | −55, 24 | |
| Week 24 | n | 39 | | 39 | | 38 | | 38 | |
| | Mean (SD) | 42.4 | (34.0) | 0.6 | (17.5) | 28.5 | (16.3) | −13.5 | (17.8) |
| | Median | 35 | | 0 | | 23.5 | | −7.5 | |
| | Min, Max | 16, 212 | | −43, 65 | | 7, 91 | | −60, 21 | |
| Endpoint | n | 43 | | 43 | | 42 | | 42 | |
| | Mean (SD) | 43.9 | (34.1) | −0.3 | (20.6) | 31.0 | (24.3) | −12.1 | (17.6) |
| | Median | 35 | | −1 | | 23.5 | | −4 | |
| | Min, Max | 16, 212 | | −66, 65 | | 7, 149 | | −60, 21 | |
| AST (U/L) | | | | | | | | | |
| Baseline | n | 43 | | | | 42 | | | |
| | Mean (SD) | 34.6 | (21.1) | | | 32.4 | (16.0) | | |
| | Median | 30 | | | | 30 | | | |
| | Min, Max | 12, 145 | | | | 12, 99 | | | |
| Week 2 | n | 43 | | 43 | | 40 | | 40 | |
| | Mean (SD) | 36.3 | (26.9) | 1.7 | (13.4) | 29.0 | (14.8) | −3.6, | (8.4) |
| | Median | 29 | | 0 | | 24.5 | | −3 | |
| | Min, Max | 16, 186 | | −28, 41 | | 11, 78 | | −21, 22 | |
| Week 4 | n | 43 | | 43 | | 40 | | 40 | |
| | Mean (SD) | 41.0 | (24.9) | 6.3 | (15.3) | 29.1 | (14.4) | −3.5 | (10.7) |
| | Median | 34 | | 3 | | 26.5 | | −5 | |
| | Min, Max | 11, 148 | | −19, 59 | | 13, 89 | | −31, 43 | |
| Week 8 | n | 41 | | 41 | | 40 | | 40 | |
| | Mean (SD) | 39.6 | (29.4) | 5.1 | (14.3) | 27.5 | (12.6) | −5.1 | (10.5) |
| | Median | 30 | | 2 | | 23.5 | | −5 | |
| | Min, Max | 16, 191 | | −25, 46 | | 8, 63 | | −46, 17 | |
| Week 12 | n | 33 | | 33 | | 34 | | 34 | |
| | Mean (SD) | 39.1 | (35.1) | 3.5 | (16.0) | 28.4 | (12.0) | −4.2 | (11.9) |
| | Median | 29 | | 0 | | 25 | | −5 | |
| | Min, Max | 13, 201 | | −27, 58 | | 9, 60 | | −39, 34 | |
| Week 16 | n | 40 | | 40 | | 38 | | 38 | |
| | Mean (SD) | 39.7 | (30.9) | 4.8 | (17.1) | 29.8 | (13.6) | −3.3 | (10.8) |
| | Median | 31 | | 1 | | 26 | | −3 | |
| | Min, Max | 17, 186 | | −32, 54 | | 9, 65 | | −42, 20 | |
| Week 20 | n | 40 | | 40 | | 38 | | 38 | |
| | Mean (SD) | 35.7 | (22.8) | 0.7 | (9.8) | 28.4 | (14.5) | −4.2 | (8.4) |
| | Median | 29 | | −1 | | 24.5 | | −2.5 | |
| | Min, Max | 15, 148 | | −18, 30 | | 10, 77 | | −38, 10 | |
| Week 24 | n | 39 | | 39 | | 38 | | 38 | |
| | Mean (SD) | 37.3 | (28.7) | 2.8 | (23.4) | 25.8 | (12.2) | −5.8 | (10.4) |
| | Median | 28 | | 1 | | 23 | | −5 | |
| | Min, Max | 17, 135 | | −31, 98 | | 11, 73 | | −47, 14 | |
| Endpoint | n | 43 | | 43 | | 42 | | 42 | |
| | Mean (SD) | 38.3 | (28.7) | 3.7 | (24.0) | 27.0 | (14.0) | −5.4 | (9.9) |
| | Median | 28 | | 1 | | 24 | | −3 | |
| | Min, Max | 17, 135 | | −31, 98 | | 11, 77 | | −47, 14 | |
| Bilirubin (umol/L) | | | | | | | | | |
| Baseline | n | 43 | | | | 42 | | | |
| | Mean (SD) | 9.95 | (7.80) | | | 12.20 | (9.80) | | |
| | Median | 8.5 | | | | 9.05 | | | |
| | Min, Max | 1.7, 39.7 | | | | 2.8, 57.4 | | | |
| Week 2 | n | 43 | | 43 | | 40 | | 40 | |
| | Mean (SD) | 10.97 | (9.82) | 1.02 | (4.28) | 10.29 | (9.46) | −2.12 | (4.58) |
| | Median | 8.2 | | 0.2 | | 7.65 | | −1.85 | |
| | Min, Max | 3.4, 55.4 | | −8.9, 15.7 | | 2.4, 57.4 | | −18.2, 8.6 | |
| Week 4 | n | 43 | | 43 | | 40 | | 40 | |
| | Mean (SD) | 11.60 | (10.62) | 1.65 | (4.67) | 11.31 | (8.52) | −1.10 | (4.28) |
| | Median | 8.6 | | 0.5 | | 9.55 | | −1.5 | |
| | Min, Max | 1.7, 58.9 | | −6.8, 19.2 | | 3.4, 54.0 | | −14.2, 9.0 | |
| Week 8 | n | 41 | | 41 | | 40 | | 40 | |
| | Mean (SD) | 12.82 | (13.19) | 2.60 | (7.01) | 10.81 | (9.74) | −1.60 | (4.48) |
| | Median | 8.6 | | 1.2 | | 7.75 | | −0.95 | |
| | Min, Max | 3.4, 80.5 | | −5.2, 40.8 | | 3.4, 61.8 | | −14.9, 6.8 | |
| Week 12 | n | 33 | | 33 | | 34 | | 34 | |
| | Mean (SD) | 14.32 | (22.81) | 3.95 | (16.41) | 10.57 | (9.72) | −1.54 | (4.45) |
| | Median | 8.6 | | 0 | | 8.35 | | −0.05 | |
| | Min, Max | 1.7, 132.2 | | −5.0, 92.5 | | 3.2, 57.8 | | −13.2, 5.2 | |
| Week 16 | n | 40 | | 40 | | 38 | | 38 | |
| | Mean (SD) | 12.95 | (18.10) | 2.73 | (12.44) | 9.40 | (5.31) | −3.41 | (7.25) |
| | Median | 7.75 | | 1.05 | | 8.1 | | −1.75 | |
| | Min, Max | 3.4, 115.4 | | −8.6, 75.7 | | 3.4, 27.4 | | −37.7, 5.2 | |
| Week 20 | n | 40 | | 40 | | 38 | | 38 | |
| | Mean (SD) | 13.64 | (17.22) | 3.42 | (11.28) | 9.88 | (6.38) | −2.76 | (4.82) |
| | Median | 7.7 | | 1 | | 7.85 | | −1.7 | |
| | Min, Max | 3.4, 106.3 | | −6.8, 66.6 | | 3.7, 31.9 | | −25.5, 2.8 | |
| Week 24 | n | 39 | | 39 | | 38 | | 38 | |
| | Mean (SD) | 13.51 | (14.75) | 3.42 | (8.68) | 8.64 | (6.11) | −3.68 | (5.53) |
| | Median | 8.9 | | 1.6 | | 6.8 | | −2.35 | |
| | Min, Max | 2.9, 76.9 | | −7.6, 37.2 | | 2.5, 31.3 | | −26.1, 3.3 | |
| Endpoint | n | 43 | | 43 | | 42 | | 42 | |
| | Mean (SD) | 13.26 | (14.31) | 3.30 | (8.41) | 8.87 | (6.14) | −3.33 | (5.40) |
| | Median | 8.6 | | 1.6 | | 6.95 | | −1.95 | |
| | Min, Max | 2.9, 76.9 | | −7.6, 37.2 | | 2.5, 31.3 | | −26.1, 3.4 | |
| Gamma Glutamyl | | | | | | | | | |
| Transferase (U/L) | | | | | | | | | |
| Baseline | n | 43 | | | | 42 | | | |
| | Mean (SD) | 85.7 | (77.9) | | | 75.1 | (68.5) | | |
| | Median | 61 | | | | 38.5 | | | |
| | Min, Max | 12, 377 | | | | 8, 255 | | | |
| Week 2 | n | 43 | | 43 | | 40 | | 40 | |
| | Mean (SD) | 84.0 | (81.5) | −1.7 | (20.9) | 84.6 | (70.1) | 7.4 | (30.5) |
| | Median | 45 | | −1 | | 52.5 | | 2.5 | |
| | Min, Max | 11, 426 | | −72, 49 | | 11, 279 | | −48, 117 | |
| Week 4 | n | 43 | | 43 | | 40 | | 40 | |
| | Mean (SD) | 90.8 | (94.9) | 5.1 | (28.0) | 78.9 | (71.6) | 1.7 | (30.2) |
| | Median | 53 | | 1 | | 53.5 | | −0.5 | |
| | Min, Max | 13, 461 | | −66, 99 | | 12, 309 | | −88, 105 | |
| Week 8 | n | 41 | | 41 | | 40 | | 40 | |
| | Mean (SD) | 89.7 | (94.4) | 3.2 | (30.3) | 71.5 | (68.2) | −5.7 | (34.5) |
| | Median | 57 | | 0 | | 46 | | −5.5 | |
| | Min, Max | 10, 481 | | −78, 104 | | 6, 281 | | −145, 77 | |
| Week 12 | n | 33 | | 33 | | 34 | | 34 | |
| | Mean (SD) | 90.0 | (101.7) | −3.0 | (42.1) | 67.9 | (60.4) | −8.1 | (41.3) |
| | Median | 53 | | −5 | | 51 | | −4 | |
| | Min, Max | 11, 556 | | −75, 179 | | 8, 240 | | −162, 56 | |
| Week 16 | n | 40 | | 40 | | 38 | | 38 | |
| | Mean (SD) | 88.3 | (89.3) | 0.0 | (34.9) | 72.4 | (67.0) | −7.0 | (40.7) |
| | Median | 57.5 | | 0 | | 54 | | −5.5 | |
| | Min, Max | 12, 503 | | −74, 126 | | 6, 294 | | −161, 90 | |
| Week 20 | n | 40 | | 40 | | 38 | | 38 | |
| | Mean (SD) | 89.8 | (84.2) | 1.5 | (39.0) | 70.1 | (64.2) | −9.6 | (37.5) |
| | Median | 53 | | −0.5 | | 47.5 | | −8 | |
| | Min, Max | 16, 451 | | −79, 102 | | 8, 261 | | −146, 57 | |
| Week 24 | n | 39 | | 39 | | 38 | | 38 | |
| | Mean (SD) | 83.0 | (83.8) | −3.2 | (34.5) | 60.4 | (57.9) | −13.6 | (36.9) |
| | Median | 45 | | 0 | | 40.5 | | −6 | |
| | Min, Max | 10, 461 | | −97, 84 | | 7, 228 | | −144, 46 | |
| Endpoint | n | 43 | | 43 | | 42 | | 42 | |
| | Mean (SD) | 81.1 | (80.6) | −4.6 | (34.3) | 62.3 | (59.3) | −12.8 | (37.0) |

| | | | | |
|---|---|---|---|---|
| Median | 46 | −1 | 42 | −6 |
| Min, Max | 10, 461 | −97, 84 | 7, 228 | −144, 46 |

Note:
Baseline is defined as the last assessment prior to the start of treatment. Endpoint is defined as the last assessment after the start of treatment.

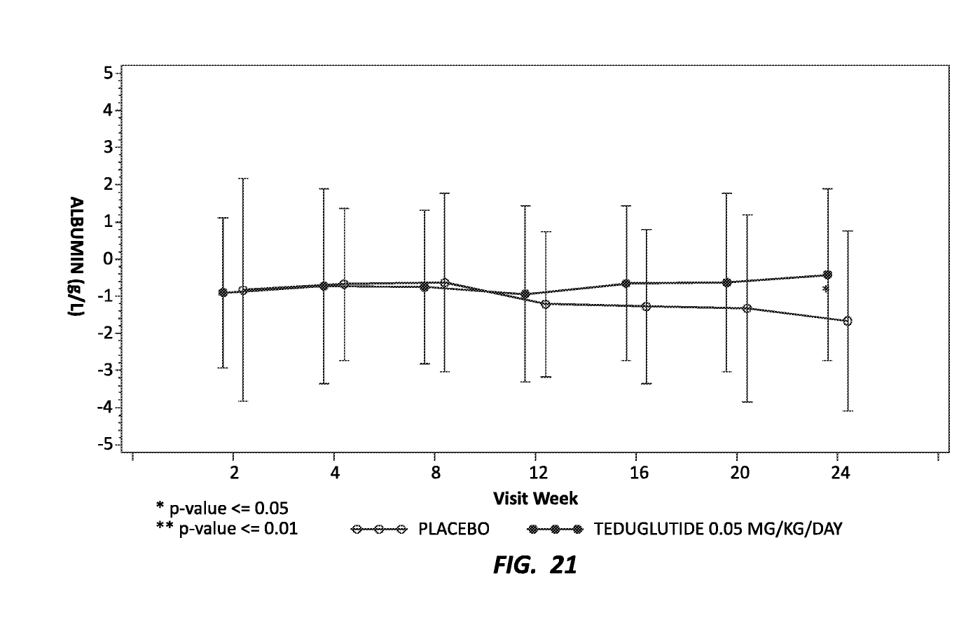

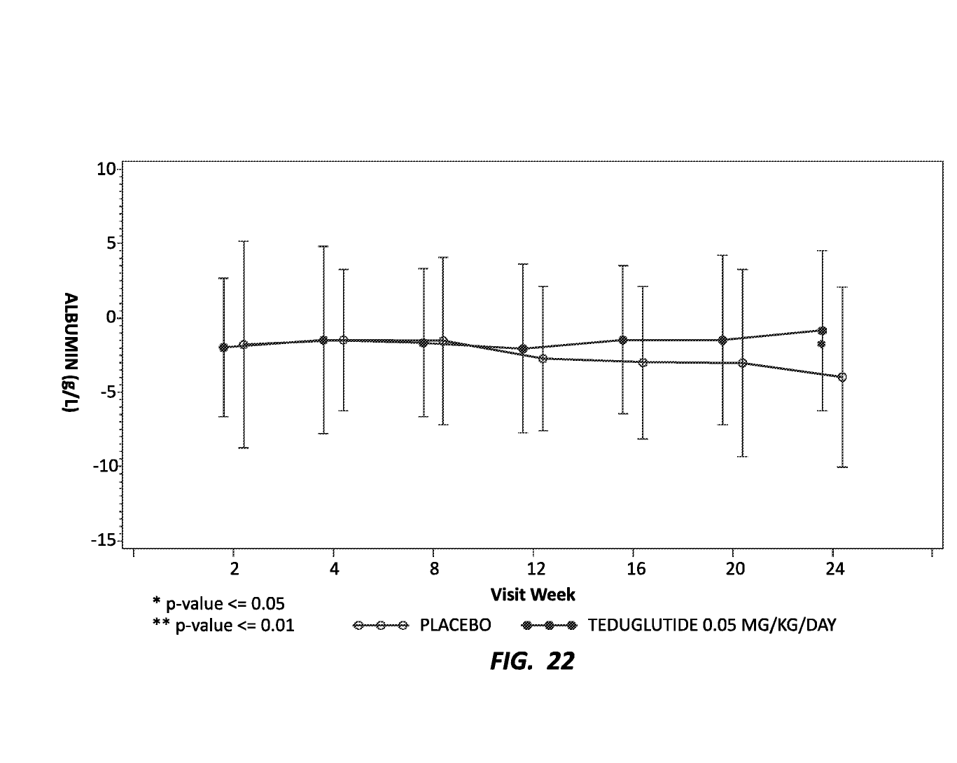

As depicted in Table 3 and FIGS. 17 through 20 and 23 through 28, in general, the cohort receiving teduglutide experienced a lower level, on average, of the tested diagnostic biomarkers elevated in individuals with liver dysfunction than the cohort receiving placebo. Furthermore, depicted in FIGS. 21 and 22, as treatment progressed, the measured level of albumin became elevated in the cohort receiving teduglutide relative to the cohort receiving placebo. All of these data are consistent with increased liver function in the cohort receiving teduglutide.

To further confirm these results, the change in each biomarker for each individual patient was sorted based on whether the biomarker increased more than 10%, decreased more than 10%, or remained within plus or minus 10% from baseline at each time point throughout the course of the study. The data for this sorting is reproduced in Table 4 below. P values are provided, which were calculated for each distribution of biomarker changes for each week using the chi-squared test.

TABLE 4
Selected Lab Tests Change from Baseline

| | | Placebo | | 0.05 mg/kg/d | |
|---|---|---|---|---|---|
| Parameter/Visit | Statistic | (N = 43) | | (N = 42) | |
| Albumin | | | | | |
| Week 2 | m | 43 | | 40 | |
| >10% increase from | n (%) | 2 | (4.7%) | 0 | |
| Baseline | | | | | |
| Within 10% of Baseline | n (%) | 37 | (86.0%) | 37 | (92.5%) |
| >10% decrease from | n (%) | 4 | (9.3%) | 3 | (7.5%) |
| Baseline | | | | | |
| | p-value | | | 0.622 | |
| Week 4 | m | 43 | | 40 | |
| >10% increase from | n (%) | 0 | | 2 | (5.0%) |
| Baseline | | | | | |
| Within 10% of Baseline | n (%) | 42 | (97.7%) | 35 | (87.5%) |
| >10% decrease from | n (%) | 1 | (2.3%) | 3 | (7.5%) |
| Baseline | | | | | |
| | p-value | | | 0.167 | |
| Week 8 | m | 41 | | 40 | |
| >10% increase from | n (%) | 1 | (2.4%) | 1 | (2.5%) |
| Baseline | | | | | |
| Within 10% of Baseline | n (%) | 39 | (95.1%) | 38 | (95.0%) |
| >10% decrease from | n (%) | 1 | (2.4%) | 1 | (2.5%) |
| Baseline | | | | | |
| | p-value | | | >0.999 | |
| Week 12 | m | 33 | | 34 | |
| >10% increase from | n (%) | 1 | (3.0%) | 1 | (2.9%) |
| Baseline | | | | | |
| Within 10% of Baseline | n (%) | 31 | (93.9%) | 31 | (91.2%) |
| >10% decrease from | n (%) | 1 | (3.0%) | 2 | (5.9%) |
| Baseline | | | | | |
| | p-value | | | >0.999 | |
| Week 16 | m | 40 | | 38 | |
| >10% increase from | n (%) | 0 | | 0 | |
| Baseline | | | | | |
| Within 10% of Baseline | n (%) | 36 | (90.0%) | 36 | (94.7%) |
| >10% decrease from | n (%) | 4 | (10.0%) | 2 | (5.3%) |
| Baseline | | | | | |
| | p-value | | | 0.676 | |
| Week 20 | m | 40 | | 38 | |
| >10% increase from | n (%) | 1 | (2.5%) | 0 | |
| Baseline | | | | | |
| Within 10% of Baseline | n (%) | 34 | (85.0%) | 37 | (97.4%) |
| >10% decrease from | n (%) | 5 | (12.5%) | 1 | (2.6%) |
| Baseline | | | | | |
| | p-value | | | 0.151 | |
| Week 24 | m | 39 | | 38 | |
| >10% increase from | n (%) | 1 | (2.6%) | 0 | |
| Baseline | | | | | |
| Within 10% of Baseline | n (%) | 33 | (84.6%) | 36 | (94.7%) |
| >10% decrease from | n (%) | 5 | (12.8%) | 2 | (5.3%) |
| Baseline | | | | | |
| | p-value | | | 0.344 | |
| Endpoint | m | 43 | | 42 | |
| >10% increase from | n (%) | 1 | (2.3%) | 0 | |
| Baseline | | | | | |
| Within 10% of Baseline | n (%) | 37 | (86.0%) | 40 | (95.2%) |
| >10% decrease from | n (%) | 5 | (11.6%) | 2 | (4.8%) |
| Baseline | | | | | |
| | p-value | | | 0.347 | |
| Alkaline Phosphatase | | | | | |
| Week 2 | m | 43 | | 40 | |
| >10% increase from | n (%) | 4 | (9.3%) | 6 | (15.0%) |
| Baseline | | | | | |
| Within 10% of Baseline | n (%) | 28 | (65.1%) | 19 | (47.5%) |
| >10% decrease from | n (%) | 11 | (25.6%) | 15 | (37.5%) |
| Baseline | | | | | |
| | p-value | | | 0.277 | |
| Week 4 | m | 42 | | 40 | |
| >10% increase from | n (%) | 8 | (19.0%) | 2 | (5.0%) |
| Baseline | | | | | |
| Within 10% of Baseline | n (%) | 25 | (59.5%) | 16 | (40.0%) |
| >10% decrease from | n (%) | 9 | (21.4%) | 22 | (55.0%) |
| Baseline | | | | | |
| | p-value | | | 0.004** | |
| Week 8 | m | 40 | | 40 | |
| >10% increase from | n (%) | 13 | (32.5%) | 2 | (5.0%) |
| Baseline | | | | | |
| Within 10% of Baseline | n (%) | 15 | (37.5%) | 13 | (32.5%) |
| >10% decrease from | n (%) | 12 | (30.0%) | 25 | (62.5%) |
| Baseline | | | | | |
| | p-value | | | 0.001** | |
| Week 12 | m | 33 | | 34 | |
| >10% increase from | n (%) | 8 | (24.2%) | 5 | (14.7%) |
| Baseline | | | | | |
| Within 10% of Baseline | n (%) | 12 | (36.4%) | 6 | (17.6%) |
| >10% decrease from | n (%) | 13 | (39.4%) | 23 | (67.6%) |
| Baseline | | | | | |
| | p-value | | | 0.069 | |
| Week 16 | m | 40 | | 38 | |
| >10% increase from | n (%) | 11 | (27.5%) | 8 | (21.1%) |
| Baseline | | | | | |
| Within 10% of Baseline | n (%) | 14 | (35.0%) | 7 | (18.4%) |
| >10% decrease from | n (%) | 15 | (37.5%) | 23 | (60.5%) |
| Baseline | | | | | |
| | p-value | | | 0.11 | |
| Week 20 | m | 40 | | 38 | |
| >10% increase from | n (%) | 10 | (25.0%) | 3 | (7.9%) |
| Baseline | | | | | |
| Within 10% of Baseline | n (%) | 14 | (35.0%) | 12 | (31.6%) |
| >10% decrease from | n (%) | 16 | (40.0%) | 23 | (60.5%) |
| Baseline | | | | | |
| | p-value | | | 0.09 | |
| Week 24 | m | 39 | | 38 | |
| >10% increase from | n (%) | 13 | (33.3%) | 6 | (15.8%) |
| Baseline | | | | | |
| Within 10% of Baseline | n (%) | 10 | (25.6%) | 9 | (23.7%) |
| >10% decrease from | n (%) | 16 | (41.0%) | 23 | (60.5%) |
| Baseline | | | | | |
| | p-value | | | 0.159 | |
| Endpoint | m | 43 | | 42 | |
| >10% increase from | n (%) | 13 | (30.2%) | 6 | (14.3%) |
| Baseline | | | | | |
| Within 10% of Baseline | n (%) | 13 | (30.2%) | 12 | (28.6%) |
| >10% decrease from | n (%) | 71 | (39.5%) | 24 | (57.1%) |
| Baseline | | | | | |
| | p-value | | | 0.148 | |
| ALT | | | | | |
| Week 2 | m | 43 | | 40 | |
| >10% increase from | n (%) | 15 | (34.9%) | 7 | (17.5%) |
| Baseline | | | | | |
| Within 10% of Baseline | n (%) | 11 | (25.6%) | 10 | (25.0%) |
| >10% decrease from | n (%) | 17 | (39.5%) | 23 | (57.5%) |
| Baseline | | | | | |
| | p-value | | | 0.17 | |
| Week 4 | m | 43 | | 40 | |
| >10% increase from | n (%) | 20 | (46.5%) | 6 | (15.0%) |
| Baseline | | | | | |
| Within 10% of Baseline | n (%) | 9 | (20.9%) | 8 | (20.0%) |
| >10% decrease from | n (%) | 14 | (32.6%) | 26 | (65.0%) |
| Baseline | | | | | |
| | p-value | | | 0.004** | |
| Week 8 | m | 41 | | 40 | |
| >10% increase from | n (%) | 19 | (46.3%) | 7 | (17.5%) |
| Baseline | | | | | |
| Within 10% of Baseline | n (%) | 9 | (22.0%) | 7 | (17.5%) |
| >10% decrease from | n (%) | 13 | (31.7%) | 26 | (65.0%) |
| Baseline | | | | | |
| | p-value | | | 0.006** | |
| Week 12 | m | 33 | | 34 | |
| >10% increase from | n (%) | 11 | (33.3%) | 7 | (20.6%) |
| Baseline | | | | | |
| Within 10% of Baseline | n (%) | 13 | (39.4%) | 10 | (29.4%) |
| >10% decrease from | n (%) | 9 | (27.3%) | 17 | (50.0%) |
| Baseline | | | | | |
| | p-value | | | 0.153 | |
| Week 16 | m | 40 | | 38 | |
| >10% increase from | n (%) | 17 | (42.5%) | 8 | (21.1%) |
| Baseline | | | | | |
| Within 10% of Baseline | n (%) | 10 | (25.0%) | 10 | (26.3%) |
| >10% decrease from | n (%) | 13 | (32.5%) | 20 | (52.6%) |
| Baseline | | | | | |
| | p-value | | | 0.1 | |
| Week 20 | m | 40 | | 38 | |
| >10% increase from | n (%) | 16 | (40.0%) | 5 | (13.2%) |
| Baseline | | | | | |
| Within 10% of Baseline | n (%) | 10 | (25.0%) | 10 | (26.3%) |
| >10% decrease from | n (%) | 14 | (35.0%) | 23 | (60.5%) |
| Baseline | | | | | |
| | p-value | | | 0.017* | |
| Week 24 | m | 39 | | 38 | |
| >10% increase from | n (%) | 14 | (35.9%) | 4 | (10.5%) |
| Baseline | | | | | |
| Within 10% of Baseline | n (%) | 13 | (33.3%) | 10 | (26.3%) |
| >10% decrease from | n (%) | 12 | (30.8%) | 24 | (63.2%) |
| Baseline | | | | | |
| | p-value | | | 0.007** | |
| Endpoint | m | 43 | | 42 | |
| >10% increase from | n (%) | 15 | (34.9%) | 5 | (11.9%) |
| Baseline | | | | | |
| Within 10% of Baseline | n (%) | 14 | (32.6%) | 12 | (28.6%) |
| >10% decrease from | n (%) | 14 | (32.6%) | 25 | (59.5%) |
| Baseline | | | | | |
| | p-value | | | 0.017* | |
| AST | | | | | |
| Week 2 | m | 43 | | 40 | |
| >10% increase from | n (%) | 17 | (39.5%) | 5 | (12.5%) |
| Baseline | | | | | |
| Within 10% of Baseline | n (%) | 14 | (32.6%) | 15 | (37.5%) |
| >10% decrease from | n (%) | 12 | (27.9%) | 20 | (50.0%) |
| Baseline | | | | | |
| | p-value | | | 0.014* | |
| Week 4 | m | 43 | | 40 | |
| >10% increase from | n (%) | 19 | (44.2%) | 8 | (20.0%) |
| Baseline | | | | | |
| Within 10% of Baseline | n (%) | 15 | (34.9%) | 6 | (15.0%) |
| >10% decrease from | n (%) | 9 | (20.9%) | 26 | (65.0%) |
| Baseline | | | | | |
| | p-value | | | <.001** | |
| Week 8 | m | 41 | | 40 | |
| >10% increase from | n (%) | 20 | (48.8%) | 8 | (20.0%) |
| Baseline | | | | | |
| Within 10% of Baseline | n (%) | 11 | (26.8%) | 8 | (20.0%) |
| >10% decrease from | n (%) | 10 | (24.4%) | 24 | (60.0%) |
| Baseline | | | | | |
| | p-value | | | 0.003** | |
| Week 12 | m | 33 | | 34 | |
| >10% increase from | n (%) | 11 | (33.3%) | 7 | (20.6%) |
| Baseline | | | | | |
| Within 10% of Baseline | n (%) | 16 | (48.5%) | 6 | (17.6%) |
| >10% decrease from | n (%) | 6 | (18.2%) | 21 | (61.8%) |
| Baseline | | | | | |
| | p-value | | | <.001** | |
| Week 16 | m | 40 | | 38 | |
| >10% increase from | n (%) | 16 | (40.0%) | 7 | (18.4%) |
| Baseline | | | | | |
| Within 10% of Baseline | n (%) | 12 | (30.0%) | 12 | (31.6%) |
| >10% decrease from | n (%) | 12 | (30.0%) | 19 | (50.0%) |
| Baseline | | | | | |
| | p-value | | | 0.085 | |
| Week 20 | m | 40 | | 38 | |
| >10% increase from | n (%) | 13 | (32.5%) | 7 | (18.4%) |
| Baseline | | | | | |
| Within 10% of Baseline | n (%) | 15 | (37.5%) | 11 | (28.9%) |
| >10% decrease from | n (%) | 12 | (30.0%) | 20 | (52.6%) |
| Baseline | | | | | |
| | p-value | | | 0.122 | |
| Week 24 | m | 39 | | 38 | |
| >10% increase from | n (%) | 13 | (33.3%) | 6 | (15.8%) |
| Baseline | | | | | |
| Within 10% of Baseline | n (%) | 14 | (35.9%) | 8 | (21.1%) |
| >10% decrease from | n (%) | 12 | (30.8%) | 24 | (63.2%) |
| Baseline | | | | | |
| | p-value | | | 0.019* | |
| Endpoint | m | 43 | | 42 | |
| >10% increase from | n (%) | 14 | (32.6%) | 6 | (14.3%) |
| Baseline | | | | | |
| Within 10% of Baseline | n (%) | 16 | (37.2%) | 11 | (26.2%) |
| >10% decrease from | n (%) | 13 | (30.2%) | 25 | (59.5%) |
| Baseline | | | | | |
| | p-value | | | 0.018* | |
| Bilirubin | | | | | |
| Week 2 | m | 43 | | 40 | |
| >10% increase from | n (%) | 19 | (44.2%) | 7 | (17.5%) |
| Baseline | | | | | |
| Within 10% of Baseline | n (%) | 12 | (27.9%) | 5 | (12.5%) |
| >10% decrease from | n (%) | 12 | (27.9%) | 28 | (70.0%) |
| Baseline | | | | | |
| | p-value | | | <.001** | |
| Week 4 | m | 43 | | 40 | |
| >10% increase from | n (%) | 18 | (41.9%) | 14 | (35.0%) |
| Baseline | | | | | |
| Within 10% of Baseline | n (%) | 16 | (37.2%) | 7 | (17.5%) |
| >10% decrease from | n (%) | 9 | (20.9%) | 19 | (47.5%) |
| Baseline | | | | | |
| | p-value | | | 0.022* | |
| Week 8 | m | 41 | | 40 | |
| >10% increase from | n (%) | 24 | (58.5%) | 10 | (25.0%) |
| Baseline | | | | | |
| Within 10% of Baseline | n (%) | 10 | (24.4%) | 10 | (25.0%) |
| >10% decrease from | n (%) | 7 | (17.1%) | 20 | (50.0%) |
| Baseline | | | | | |
| | p-value | | | 0.002** | |
| Week 12 | m | 33 | | 34 | |
| >10% increase from | n (%) | 14 | (42.4%) | 9 | (26.5%) |
| Baseline | | | | | |
| Within 10% of Baseline | n (%) | 10 | (30.3%) | 10 | (29.4%) |
| >10% decrease from | n (%) | 9 | (27.3%) | 15 | (44.1%) |
| Baseline | | | | | |
| | p-value | | | 0.297 | |
| Week 16 | m | 40 | | 38 | |
| >10% increase from | n (%) | 21 | (52.5%) | 9 | (23.7%) |
| Baseline | | | | | |
| Within 10% of Baseline | n (%) | 11 | (27.5%) | 5 | (13.2%) |
| >10% decrease from | n (%) | 8 | (20.0%) | 24 | (63.2%) |
| Baseline | | | | | |
| | p-value | | | <.001** | |
| Week 20 | m | 40 | | 38 | |
| >10% increase from | n (%) | 21 | (52.5%) | 8 | (21.1%) |
| Baseline | | | | | |
| Within 10% of Baseline | n (%) | 6 | (15.0%) | 5 | (13.2%) |
| >10% decrease from | n (%) | 13 | (32.5%) | 25 | (65.8%) |
| Baseline | | | | | |
| | p-value | | | 0.007** | |
| Week 24 | m | 39 | | 38 | |
| >10% increase from | n (%) | 22 | (56.4%) | 6 | (15.8%) |
| Baseline | | | | | |
| Within 10% of Baseline | n (%) | 6 | (15.4%) | 6 | (15.8%) |
| >10% decrease from | n (%) | 11 | (28.2%) | 26 | (68.4%) |
| Baseline | | | | | |
| | p-value | | | <.001** | |
| Endpoint | m | 43 | | 42 | |
| >10% increase from | n (%) | 24 | (55.8%) | 7 | (16.7%) |
| Baseline | | | | | |
| Within 10% of Baseline | n (%) | 7 | (16.3%) | 8 | (19.0%) |
| >10% decrease from | n (%) | 12 | (27.9%) | 27 | (64.3%) |
| Baseline | | | | | |

| | |
|---|---|
| p-value | <.001** |

*p <= 0.050;
**p <= 0.010.
Note:
Percentages are based upon m, defined as the number of subjects in the Safety Population who have both a baseline and post-baseline visit value for the associated parameter.
Note:
Baseline is defined as the last assessment prior to the start of treatment. Endpoints defined as the last assessment after the start of treatment.
Note:
The treatment comparison is based on an exact chi-square test.

These data confirm the results from Example 1, namely that, in general, an individual receiving teduglutide is more likely to experience a>10% decrease in liver biomarkers elevated in individuals with liver dysfunction than an individual receiving placebo alone. Conversely, an individual receiving teduglutide is also less likely to experience a>10% increase in biomarkers elevated in individuals with liver dysfunction than an individual receiving placebo alone. These results support the conclusion that teduglutide is useful to both treat and protect against liver dysfunction.

### Example 3

In this example the data presented in Example 2 are analyzed to evaluate whether the beneficial effects of teduglutide on liver function tests are related to the reduction of parenteral nutrition volume.

Table 5 depicts the change in liver enzyme levels from baseline to week 24 in responders and non-responders. Responders are human patients with reduced parenteral nutrition, while non-responders are human patients without reduced parenteral nutrition. Human patients either received placebo or teduglutide at 0.05 mg/kg/day. Known biomarkers of liver function were monitored for 24 weeks. The liver biomarkers tested included ALP, ALT, AST, total bilirubin and GGTP.

TABLE 5
Change from Baseline to Week 24 in Liver Enzyme Levels

| Lab Parameter (unit) | Statistic | Responder | | Non-responder | | Total | |
|---|---|---|---|---|---|---|---|
| | | Placebo | | | | | |
| ALKALINE PHOSPHATASE (U/L) | n | 13 | | 26 | | 39 | |
| | Mean (SD) | −7.62 | (43.632) | −3.54 | (48.529) | −4.90 | (46.415) |
| | Median | −7 | | 2 | | −1 | |
| | Min, Max | −101, 59.0 | | −137, 94.0 | | −137, 94.0 | |
| ALT (U/L) | n | 13 | | 26 | | 39 | |
| | Mean (SD) | 0.54 | (9.649) | 0.58 | (20.451) | 0.56 | (17.452) |
| | Median | 2 | | −1 | | 0 | |
| | Min, Max | −16.0, 17.0 | | −43.0, 65.0 | | −43.0, 65.0 | |
| AST (U/L) | n | 13 | | 26 | | 39 | |
| | Mean (SD) | −0.54 | (5.285) | 4.54 | (28.423) | 2.85 | (23.371) |
| | Median | −1 | | 1 | | 1 | |
| | Min, Max | −9.0, 10.0 | | −31.0, 98.0 | | −31.0, 98.0 | |
| BILIRUBIN, TOTAL (μmol/L) | n | 13 | | 26 | | 39 | |
| | Mean (SD) | 2.64 | (7.458) | 3.81 | (9.344) | 3.42 | (8.679) |
| | Median | 1.7 | | 0.4 | | 1.6 | |
| | Min, Max | −4.8, 25.7 | | −7.6, 37.2 | | −7.6, 37.2 | |
| GGTP (U/L) | n | 13 | | 26 | | 39 | |
| | Mean (SD) | −15.00 | (29.858) | 2.69 | (35.681) | −3.21 | (34.503) |
| | Median | −5 | | 3.5 | | 0 | |
| | Min, Max | −96.0, 28.0 | | −97.0, 84.0 | | −97.0, 84.0 | |
| | | Teduglutide 0.05 mg/kg/day | | | | | |
| ALKALINE PHOSPHATASE (U/L) | n | 27 | | 11 | | 38 | |
| | Mean (SD) | −35.52 | (33.942) | −13.82 | (33.487) | −29.24 | (34.815) |
| | Median | −34 | | −7 | | −24.5 | |
| | Min, Max | −122, 33.0 | | −107, 12.0 | | −122, 33.0 | |
| ALT (U/L) | n | 27 | | 11 | | 38 | |
| | Mean (SD) | −17.48 | (19.380) | −3.82 | (7.209) | −13.53 | (17.815) |
| | Median | −14 | | −3 | | −7.5 | |
| | Min, Max | −60.0, 21.0 | | −23.0, 5.0 | | −60.0, 21.0 | |
| AST (U/L) | n | 27 | | 11 | | 38 | |
| | Mean (SD) | −7.30 | (11.776) | −2.18 | (4.119) | −5.82 | (10.371) |
| | Median | −7 | | −2 | | −5 | |
| | Min, Max | −47.0, 14.0 | | −10.0, 4.0 | | −47.0, 14.0 | |
| BILIRUBIN, TOTAL (μmol/L) | n | 27 | | 11 | | 38 | |
| | Mean (SD) | −4.19 | (5.998) | −2.43 | (4.151) | −3.68 | (5.531) |
| | Median | −2.4 | | −1.7 | | −2.35 | |
| | Min, Max | −26.1, 2.1 | | −11.1, 3.3 | | −26.1, 3.3 | |
| GGTP (U/L) | n | 27 | | 11 | | 38 | |
| | Mean (SD) | −18.41 | (41.808) | −1.91 | (16.861) | −13.63 | (36.914) |

| | | | |
|---|---|---|---|
| Median | −10 | 3 | −6 |
| Min, Max | −144, 46.0 | −45.0, 19.0 | −144, 46.0 |

Table 6 depicts the change in liver enzyme levels (ALP, ALT, AST, total bilirubin and GGTP), as presented in Table 5, and stratifies them by treatment group (placebo versus teduglutide at 0.05 mg/kg/day) and responder status (responder versus non-responder).

TABLE 6
Analysis of Change from Baseline to Week 24 in Liver Enzyme Levels

| | | Treatment | | | | |
|---|---|---|---|---|---|---|
| | | | | teduglutide 0.05 | | |
| Lab Parameter (unit) | Statistic | Placebo | | mg/kg/day | | p-value |
| ALKALINE PHOSPHATASE (U/L) | LS Means (SE) | −5.58 | (6.968) | −24.67 | (7.338) | 0.0632 |
| | 95% CI | −19.47, | 8.31 | −39.29, | −10.04 | |
| ALT (U/L) | LS Means (SE) | 0.56 | (2.939) | −10.65 | (3.095) | 0.0105 |
| | 95% CI | −5.30, | 6.41 | −16.82, | −4.48 | |
| AST (U/L) | LS Means (SE) | 2.00 | (3.099) | −4.74 | (3.264) | 0.1386 |
| | 95% CI | −4.18, | 8.18 | −11.24, | 1.77 | |
| BILIRUBIN, TOTAL (μmol/L) | LS Means (SE) | 3.23 | (1.251) | −3.31 | (1.317) | 0.0006 |
| | 95% CI | 0.73, | 5.72 | −5.93, | −0.68 | |
| GGTP (U/L) | LS Means (SE) | −6.15 | (5.990) | −10.16 | (6.308) | 0.6467 |
| | 95% CI | −18.09, | 5.79 | −22.73, | 2.41 | |
| | | Responder Status | | | | |
| Lab Parameter (unit) | Statistic | Responder | | Non-responder | | p-value |
| ALKALINE PHOSPHATASE (U/L) | LS Means (SE) | −21.57 | (6.925) | −8.68 | (7.379) | 0.2068 |
| | 95% CI | −35.37, | −7.76 | −23.38, | 6.03 | |
| ALT (U/L) | LS Means (SE) | −8.47 | (2.921) | −1.62 | (3.112) | 0.1128 |
| | 95% CI | −14.29, | −2.65 | −7.82, | 4.58 | |
| AST (U/L) | LS Means (SE) | −3.92 | (3.080) | 1.18 | (3.282) | 0.2612 |
| | 95% CI | −10.06, | 2.22 | −5.36, | 7.72 | |
| BILIRUBIN, TOTAL (μmol/L) | LS Means (SE) | −0.77 | (1.243) | 0.69 | (1.324) | 0.4223 |
| | 95% CI | −3.25, | 1.70 | −1.95, | 3.33 | |
| GGTP (U/L) | LS Means (SE) | −16.70 | (5.953) | 0.39 | (6.343) | 0.0532 |
| | 95% CI | −28.57, | −4.84 | −12.25, | 13.03 | |

The data presented in this example show that the positive effect on liver function tests is an effect of teduglutide (difference from placebo), and that the major part of the effect is not mediated through the reduction of parenteral nutrition (difference between responders and non-responders).

It will be apparent to those having skill in the art that many changes may be made to the details of the above-described embodiments without departing from the underlying principles of the invention. The scope of the present invention should, therefore, be determined only by the following claims.

1. A method of treating impaired liver function in an individual experiencing intestinal failure, short bowel syndrome, or parenteral nutrition, comprising the step of administering to an individual having impaired liver function one or more of a GLP-2 peptide or a GLP-2 peptide analog in an amount effective to cause improvement in liver function.

2. The method of claim 1, wherein the GLP-2 peptide analog is teduglutide (SEQ ID NO.:4).

3. The method of claim 1, wherein the GLP-2 peptide or the GLP-2 peptide analog is administered at a dose of between about 0.001 mg/kg/day and about 10 mg/kg/day.

4. The method of claim 2, wherein teduglutide is administered at a dose of between about 0.05 mg/kg/day and about 0.1 mg/kg/day.

5. The method of claim 1 wherein the improvement in liver function is observed within about four weeks after the beginning of administering the GLP-2 peptide or GLP-2 peptide analog to the individual.

6. The method of claim 1 wherein the improvement in liver function is monitored by the use of one or more diagnostic biomarkers.

7. The method of claim 6, wherein the diagnostic biomarkers are selected from the group consisting of: bilirubin, gamma glutamyl transferase, alanine transaminase, aspartate aminotransferase, alkaline phosphatase and albumin.

8. The method of claim 7, wherein the individual level of bilirubin, gamma glutamyl transferase, alanine transaminase, aspartate aminotransferase or alkaline phosphatase, if selected, decreases at least about 5 percent or wherein the level of albumin, if selected, increases at least about 5 percent.

9. A method for prophylaxis against impairment of liver function in an individual experiencing intestinal failure, short bowel syndrome, or parenteral nutrition, comprising the step of administering to an individual one or more of a GLP-2 peptide or a GLP-2 peptide analog in an amount effective for prophylaxis against impaired liver function.

10. The method of claim 9, wherein the GLP-2 peptide analog is teduglutide (SEQ ID NO.:4).

11. The method of claim 9, wherein the GLP-2 peptide or the GLP-2 peptide analog is administered at a dose of between about 0.001 mg/kg/day and about 10 mg/kg/day.

12. The method of claim 10, wherein teduglutide is administered at a dose of between about 0.05 mg/kg/day and about 0.1 mg/kg/day.

13. The method of claim 9 wherein the prophylaxis against impaired liver function is observed within about four weeks after the beginning of administering the GLP-2 peptide or GLP-2 peptide analog to the individual.

14. The method of claim 9 wherein the prophylaxis against impaired liver function is monitored by the use of one or more diagnostic biomarkers.

15. The method of claim 14, wherein the diagnostic biomarkers are selected from the group consisting of: bilirubin, gamma glutamyl transferase, alanine transaminase, aspartate aminotransferase, alkaline phosphatase and albumin.

16. The method of claim 15, wherein the individual level of bilirubin, gamma glutamyl transferase, alanine transaminase, aspartate aminotransferase or alkaline phosphatase, if selected, increases, if at all, less than about 10 percent or wherein the level of albumin, if selected, decreases, if at all, less than about 10 percent.


SEQUENCE LISTING (4 sequences)

SEQ ID NO: 1 - protein, 35 aa, Artificial Sequence
  GLP-2 peptide consensus sequence
XHADGSFSDEMNTXLAXLAXRDFINWLXXTKITDX

SEQ ID NO: 2 - protein, 28 aa, Artificial Sequence
  GLP-2 peptide analog consensus sequence
XXXXXSFSDXLDNLAXXDFXWLIQTKXX

SEQ ID NO: 3 - protein, 33 aa, Artificial Sequence
  Human GLP-2 (1-33)
HADGSFSDEMNTILDNLAARDFINWLIQTKITD

SEQ ID NO: 4 - protein, 33 aa, Artificial Sequence
  Teduglutide
HGDGSFSDEMNTILDNLAARDFINWLIQTKITD